(19) United States
(12) Patent Application Publication
(10) Pub. No.: US 2010/0311654 A1
(43) Pub. Date: Dec. 9, 2010

(54) Modified Polysaccharide-Based Delivery of Nucleic Acids

(76) Inventors: Krishnendu Roy, Austin, TX (US); Bilal Ghosn, Seattle, WA (US); Sudhir Kasturi, Atlanta, GA (US)

Correspondence Address: BAKER BOTTS L.L.P., 2001 ROSS AVENUE, SUITE 600, DALLAS, TX, 75201-2980, US

(21) Appl. No.: 12/720,546

(22) Filed: Mar. 9, 2010

Related U.S. Application Data
(63) Continuation-in-part of application No. PCT/US2008/075799, filed on Sep. 10, 2008.
(60) Provisional application No. 60/971,070, filed on Sep. 10, 2007.

(51) Int. Cl. A61K 31/7052; A61K 47/36; A61K 38/00; A61K 31/726; A61K 38/22; C07H 21/00; C07H 1/00; C08B 37/02; C07H 5/06; A61P 9/00

(52) U.S. Cl. 514/9.7; 514/44 R; 514/777; 514/44 A; 514/1.1; 514/54; 536/23.1; 536/123.1; 536/24.5; 536/20; 536/51; 536/55.2; 977/773; 977/906

(57) ABSTRACT

The present disclosure provides compositions for enhanced delivery of therapeutic agents. Drug delivery vehicle compositions may include a modified polysaccharide (e.g. chitosan) having at least one secondary amine or at least one tertiary amine and a therapeutic agent such as a therapeutic nucleic acid and/or a therapeutic anionic agent. Various additional modifications of a modified polysaccharide of the disclosure are also described. Exemplary therapeutic agents may include but are not limited to nucleic acids, polynucleotides, siRNA and/or pDNA. Compositions of the disclosure may be formulated as nanoparticles and may provide one or more advantages including efficient transfection, bio-delivery, availability, buffering ability, serum stability. Methods for synthesizing the drug delivery compositions are also set forth. The disclosure also provides methods for delivering/administering therapeutic agents using the drug delivery compositions of the disclosure to a patient in need thereof. Therapeutic methods are also provided.

### CROSS-REFERENCE TO RELATED APPLICATIONS

This application is a continuation-in-part of and claims the benefit of PCT Application No. PCT/US2008/075799 filed on Sep. 10, 2008, entitled “ENHANCEMENT OF POLYSACCHARIDE-MEDIATED NUCLEIC ACID DELIVERY”, which is incorporated herein in its entirety.

### FIELD OF THE DISCLOSURE

The present disclosure generally relates to delivery of therapeutic agents. In particular, the present disclosure relates to drug delivery vehicle compositions and to methods of making and using such compositions for enhanced delivery of therapeutic agents such as, but not limited to, a therapeutic nucleic acid and/or an anionic therapeutic-agent. A drug delivery composition of the disclosure may be a nanoparticle. The present disclosure also relates to methods of administering/delivering a nucleic acid and/or an anionic therapeutic agent to a subject in need thereof using a drug delivery composition of the disclosure.

### BACKGROUND OF THE DISCLOSURE

From DNA vaccines for immunotherapy to gene knockdown through antisense oligonucleotides and siRNA, application of nucleic acids to more effectively treat a wide array of complex disorders are becoming more of a reality. Despite being a very powerful therapeutic agent, the efficacy of nucleic acids is limited by their serum instability, minimal cellular uptake in the native state, poor endosomal escape and inefficient cellular distribution. Over the past 15 years efforts have focused on the use of viral and non-viral carriers for delivering nucleic acids into cells. Although viral vectors are very efficient both in vitro an in vivo, substantial possibility of immunogenic, oncogenic and cytotoxic consequences are present and has thus facilitated the study of synthetic or natural biomaterials as gene carriers. These materials offer a safer alternative to viral vectors, but suffer from low transfection efficiencies.

Recent research has focused on the use of a variety of cationic molecules due to their inherent ability to complex with the negatively charged nucleic acids, providing a means for condensation and imparting a positive surface charge to the nucleic acids. These include cationic lipids as well as cationic polymers such as polyethyleneimine (PEI), dendrimers and polylysine, which have been demonstrated to be effective at enhancing transfection of the nucleic acids and retaining stability and bioactivity. However, cytoxicity and in vivo systemic toxicity issues have significantly limited human applications of these materials. Thus, development of tailored biomaterials that allow for highly efficient delivery inside cells while exhibiting minimal or acceptable toxicity is essential for the eventual clinical use of nucleic acids as drugs.

### SUMMARY

Therefore, a need has arisen for better compositions and methods for improved delivery of nucleic acids. The present disclosure provides drug delivery compositions, methods for synthesis of drug delivery compositions, and methods for enhanced delivery of therapeutic nucleic acids as well as anionic therapeutic agents to subjects.

In some embodiments, the present disclosure describes drug delivery vehicle compositions that may comprise at least one polysaccharide having at least one secondary amine or at least one tertiary amine (also referred to herein as a modified polysaccharide) and at least one therapeutic agent such as a therapeutic nucleic acid and/or a therapeutic anionic agent.

A drug delivery vehicle of the disclosure may be formulated as a nanoparticle. In some embodiments, a nanoparticle composition of the disclosure may have a diameter of less than 1000 nm.

In some embodiments, a drug delivery composition of the disclosure may further comprise one or more PEG molecules. In some embodiments, polyethylene glycol may be conjugated to a modified polysaccharide. In some embodiments, polyethylene glycol may be added to a nanoparticle composition of the disclosure. PEG molecules of various molecular weights may be used. Addition of PEG may in some embodiments increase the serum stability of a drug delivery vehicle of the disclosure.

In some embodiments, a modified polysaccharide comprised in a drug delivery composition of the disclosure may comprise one or more additional secondary amines. In some embodiments, a modified polysaccharide may comprise one or more additional tertiary amines. Accordingly, modified polysaccharides of the disclosure may be described as having a degree of substitution. The degree of substitution of a modified polysaccharide according to the disclosure may be determined by NMR characterization methods. The present disclosure describes two different NMR characterization methods to determine a degree of substitution, one in Example 1 which is by a ninhydrin assay and NMR analysis and another in Example 6 which is by NMR analysis comprising evaluation of imidazole peaks. In some embodiments, the present disclosure describes degrees of substitution as determined from the methods of Example 1 and some embodiments describe degrees of substitution as determined from the methods of Example 6. The methods of analysis used to determine the degree of substitution of a modified polysaccharide is indicated where appropriate in the application. However, in some instances measurements may be from either or both methods.

In some embodiments, the degree of substitution of a polysaccharide with at least one additional secondary amine and/or at least one tertiary amine may be at least from about 0.5% to about 99%. For example, in one embodiment the degree of substitution of a modified chitosan having at least one secondary amine or at least one tertiary amine may be at least about 0.5% as determined by NMR analysis comprising evaluation of imidazole peaks. In another example embodiment, the degree of substitution of a modified chitosan having at least one secondary amine or at least one tertiary amine may be from about 19.9% to about 30.2% as determined by ninhydrin assay and NMR analysis.

Some exemplary polysaccharides that may be used to form a drug delivery composition of the disclosure include but are not limited to chitosans, modified chitosans, modified dextrans, glucosamines, hybrid polymers of any of the preceding polymers and/or any combinations thereof.

A therapeutic agent that may be delivered using the delivery compositions of the disclosure may include one or more nucleic acids, such as but not limited to, a polynucleotide, an oligonucleotide, a DNA, a plasmid DNA (pDNA), a RNA, an antisense molecule, an siRNA, an miRNA, a DNA triple-helix or any combinations thereof. Other therapeutic agents that may be comprised in a drug delivery vehicle of the disclosure may be anionic therapeutic agents, such as but not limited to, anionic proteins, anionic biomolecules, or anionic small molecule agents, an anionic glycosaminoglycan, an anionic peptide, an anionic hormone (collectively referred to herein as an “anionic agent,” an “anionic therapeutic agent” or a “therapeutic anionic agent”).

Drug delivery vehicle compositions of the disclosure may have high transfection efficiencies. Drug delivery compositions of the disclosure may also have an effective buffering capacity in an aqueous solution from about pH 4.5 to about pH 8.5. In some embodiments, the degree of substitution of a polysaccharide with a secondary amine and/or a tertiary amine may change or alter the buffering capacity of the drug delivery vehicle. In one embodiment, an increase in the degree of substitution may increase the buffering capacity of a drug delivery composition.

In some embodiments, the degree of substitution of a polysaccharide may be related to solubility of the polysaccharide at a given pH value. For example, a modified chitosan having a 20% degree of substitution (as determined by ninhydrin assay and NMR analysis) may be at least 90% soluble at above pH 8. In some embodiments, a modified polysaccharide according to the disclosure may be at least 90%, at least 95%, at least 99% or completely soluble in an aqueous solution at a pH greater than about 7, such as the physiological pH of about 7.4.

Accordingly, drug delivery compositions of the disclosure may have or may provide one or more of the following advantages including: an increased solubility, enhanced buffering capacity in an aqueous solution, enhanced serum stability, enhanced endosomal escape properties, reduced or no biological toxicity, may facilitate cytoplasmic release of a complexed nucleic acid and/or an anionic therapeutic agent, may provide efficient transfection of a nucleic acid and/or may provide efficient delivery of an anionic therapeutic agent.

In some embodiments, secondary and/or tertiary amines may be introduced into a polysaccharide by chemical conjugation and/or a reactive compound conjugation method to obtain a modified polysaccharide or to further modify a modified polysaccharide. In some embodiments, associating or complexing a therapeutic nucleic acid or an anionic therapeutic agent may be via electrostatic interaction.

The present disclosure also relates to methods for synthesizing drug delivery compositions and may comprise: providing a polysaccharide and a reactive compound; allowing the reactive compound to react with the polysaccharide so as to introduce at least one secondary amine or at least one tertiary amine onto the polysaccharide, thereby obtaining a modified polysaccharide comprising at least one secondary amine or at least one tertiary amine; and associating or complexing one or more therapeutic nucleic acids and/or one or more therapeutic anionic agents with the modified polysaccharide, thereby forming a drug delivery composition. In some embodiments, a method for synthesizing a drug delivery composition may also comprise allowing a reactive compound to react with the polysaccharide or modified polysaccharide to add at least one additional secondary amine to the polysaccharide moiety.

A method for synthesis of a drug delivery composition may also include formulating the composition into a nanoparticle. In some embodiments nanoparticles of 1000 nm or less may be formulated. In some embodiments the method may comprise adding PEG moieties onto the drug delivery composition. In some embodiments, a method for synthesizing a drug delivery composition may comprise adding marker molecules onto the surface of the composition to facilitate organ-specific or cell-specific delivery of a therapeutic agent carried by the composition. Methods also include making pharmaceutically acceptable formulations of the drug delivery compositions suitable of in vivo administration.

The present disclosure also provides methods for delivering a therapeutic agent to a patient or subject in need thereof and may comprise: providing a pharmaceutically acceptable formulation comprising a drug delivery composition of the disclosure comprising at least one polysaccharide having at least one secondary amine or at least one tertiary amine and at least one therapeutic agent such as a therapeutic nucleic acid and/or a therapeutic anionic agent; and administering the drug delivery composition into the patient or subject. Administration may be by parenteral, oral, intranasal, topical, or other methods and additional details regarding administration and pharmaceutical formulations are provided later in this specification. The patient or subject may be a human or any mammal.

In some embodiments, a therapeutic method of the disclosure for enhanced delivery of therapeutic nucleic acids and/or anionic agents may have one or more of the following advantages including: increased solubility, enhanced buffering capacity in an aqueous solution, enhanced endosomal escape properties, reduced or no biological toxicity, reduced aggregation of nanoparticles, increased serum stability, increased bioavailability, better bio-distribution, may facilitate cytoplasmic release of a complexed nucleic acid or an anionic therapeutic agent, may provide efficient transfection of a nucleic acid, may provide efficient delivery of an anionic therapeutic agent.

Other features and advantages of the present invention will be readily apparent to those skilled in the art upon a reading of the description of the embodiments that follows.

### BRIEF DESCRIPTION OF THE DRAWINGS

Some specific example embodiments of the disclosure may be understood by referring, in part, to the following description and the accompanying drawings.

FIG. 1 shows a schematic of the carbodiimide reaction for conjugation of IAA to chitosan according to an example embodiment of the disclosure;

FIGS. 2A, 2B & 2C show NMR Spectra for unmodified chitosan (FIG. 2A), chitosan-IAA 19.9% (FIG. 2B), and chitosan-IAA 30.2% (FIG. 2C) according to an example embodiment of the disclosure;

FIGS. 3A & 3B show characteristics of modified polymers where FIG. 3A shows the buffering capacity of modified chitosan as compared to unmodified chitosan and IAA; and FIG. 3B shows the cytotoxicity of Chitosan-IAA at 0.25 mg/mL in HEK293T cells (n=3), according to an example embodiment of the disclosure;

FIGS. 4A & 4B show transfection of HEK293T cells with modified chitosan with equal weight of polymer (FIG. 4A) and various N/P ratios (FIG. 4B), wherein the three types of chitosan studied were Protasan UP CL113 (unmodified chitosan), chitosan-IAA 19.9%, and chitosan-IAA 30.2% and Exgen 500 was used as a positive control, all samples are n=3. *=p<0.05 as compared to unmodified chitosan at same N/P ratio or equivalent weight using t-test, according to an example embodiment of the disclosure;

FIG. 5 shows the transfection of GAP480 silencer siRNA and Negative Control (NC) siRNA with modified chitosans and siPORT Amine in HEK 293T cells (n=3), *=p<0.05 as compared to unmodified chitosan at same N/P ratio or equivalent weight using t-test, according to an example embodiment of the disclosure;

FIGS. 6A, 6B, 6C & 6D show synthesis, spectra and nanoparticle characteristics for drug delivery vehicles according to the disclosure, wherein FIG. 6A depicts another chemical schematic method for the synthesis of imidazole-modified chitosan (chitosan-IAA); FIG. 6B depicts the ¹H NMR Spectra of imidazole-modified chitosan showing protons of imidazole ring carbon molecules in the inset; FIG. 6C depicts effect of PEGylation on chitosan-IAA/siRNA nanoparticle size; and FIG. 6D depicts effect of PEGylation on chitosan-IAA/siRNA nanoparticle on zeta potential where nanoparticles were prepared at an N/P of 50; *=p<0.05 as compared to non-PEGylated nanoparticles using student t-test, according to one example embodiment of the disclosure;

FIGS. 7A, 7B & 7C show silencing of GAPDH protein following intravenous administration of PEGylated chitosan and chitosan-IAA/siRNA nanoparticles, wherein, FIG. 7A shows gene silencing resulting in lungs of BALB/C mice following 1 mg/kg siRNA dose; FIG. 7B shows silencing of GAPDH protein in liver of BALB/C mice with same 1 mg/kg dose; FIG. 7C shows silencing of Apolipoprotein B mRNA in the liver of BALB/C mice following intravenous administration of PEGylated chitosan-IAA/siRNA nanoparticles at various siRNA doses; GAPDH samples and ApoB samples prepared at N/P=50 and 40, respectively and *=p<0.05 as compared to control mice using one-way ANOVA, according to one example embodiment of the disclosure; and

FIGS. 8A, 8B & 8C depict silencing of GAPDH protein in lungs following intranasal administration of chitosan and chitosan-IAA/siRNA nanoparticles to BALB/C mice, wherein, FIG. 8A depicts protein expression levels following a single 0.5 mg/kg dose; FIG. 8B depicts three consecutive daily doses of 0.5 mg/kg siRNA in various formulations; and FIG. 8C depicts GAPDH expression (mRNA) following a single dose at variable concentrations (0.5, 1.0, 2.0 mg/kg siRNA) prepared with chitosan-IAA, wherein nanoparticle formulations prepared at N/P=50 and *=p<0.05 as compared to control mice using one-way ANOVA, according to one example embodiment of the disclosure.

While the present disclosure is susceptible to various modifications and alternative forms, specific example embodiments have been shown in the figures and are described in more detail below. It should be understood, however, that the description of specific example embodiments is not intended to limit the invention to the particular forms disclosed, but on the contrary, this disclosure is to cover all modifications and equivalents as illustrated, in part, by the appended claims.

### DETAILED DESCRIPTION

The present disclosure, in one embodiment, describes drug delivery vehicles for delivering therapeutic agents such as therapeutic nucleic acids and/or therapeutic anionic agents and may comprise 1) at least one polysaccharide having at least one secondary amine or at least one tertiary amine; and 2) at least one therapeutic nucleic acid and/or at least one anionic therapeutic agent. In some embodiments, a drug delivery vehicle may further comprise at least one additional secondary amine and/or at least one additional tertiary amine.

In some embodiments, a drug delivery vehicle of the disclosure may be formulated as a nanoparticle. In some embodiments, a nanoparticle comprising a drug delivery vehicle of the disclosure may be less than 1000 nm in diameter. In some embodiments, a nanoparticle according to the present disclosure may be about 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 800 nm, about 850 nm, about 900 nm, about 950 nm or about 999 nm, or any intermediate size less than 1000 nm in diameter. In some embodiments a nanoparticle composition of the disclosure may be from about 50 nm to about 250 nm in diameter. Further embodiments relating to nanoparticles are described later in the specification

Polysaccharides of the Drug Delivery Compositions

A drug delivery vehicle composition of the disclosure may comprise a modified polysaccharide having a degree of substitution with at least one secondary amine or at least one tertiary amine. In some embodiments, a modified polysaccharide may also include at least one additional secondary amine. In some embodiments, a modified polysaccharide may also include at least two secondary amines.

In some embodiments, the degree of substitution of a modified polysaccharide having at least one additional secondary amine and/or at least one tertiary amine is at least about 0.5% as determined by NMR analysis comprising evaluation of imidazole peaks (see Example 6). In some embodiments, the degree of substitution of a modified polysaccharide having at least one additional secondary amine and/or at least one tertiary amine is at least about 2% to about 3% as determined by NMR analysis comprising evaluation of imidazole peaks (see Example 6). In some embodiments, the degree of substitution of a modified polysaccharide having at least one additional secondary amine and/or at least one tertiary amine is at least about 10% as determined by ninhydrin and NMR analysis (see Example 1). In some embodiments, the degree of substitution of a modified polysaccharide having at least one additional secondary amine and/or at least one tertiary amine is from about 19.9% to at least about 30.2% as determined by ninhydrin and NMR analysis (see Example 1). In some embodiments, the degree of substitution of a modified polysaccharide having at least one additional secondary amine or at least one tertiary amine may be a range of at least from about 0.5% to about 99%, from about 0.5% to about 3%, from about 0.5% to about 5%, from about 0.5% to about 10%, from about 1% to about 3%, from about 15% to about 20%, or from about 20% to about 30%, or from about 19.9% to at least about 30.2%.

In one embodiment, drug delivery vehicle compositions of the disclosure may comprise a polysaccharide having at least one (or more) primary amine and at least one (or more) secondary amine or one (or more) tertiary amines that may be naturally present or that may be added by one or more chemical reactions in its structure. A polysaccharide comprised in a composition of the disclosure may further comprise additional secondary and/or tertiary amines (naturally or synthetically added).

In some embodiments, a drug delivery composition of this disclosure may comprise a polysaccharide having at least one primary amine that maybe naturally present in its structure and having at least one secondary amine that maybe naturally present in its structure or having at least one tertiary amine that may be naturally present in its structure. A polysaccharide (e.g., such as a polysaccharide described in the previous sentence) may be modified by the addition of at least one (more) secondary amine and/or at least one tertiary amine through covalent conjugation of other amine carrying molecules to form a drug delivery composition. In some embodiments, one or more secondary amine(s) or one or more additional secondary amine(s) and/or one or more tertiary amine(s) may be introduced into a polysaccharide by a reactive compound conjugation method. For example, carbodiimide coupling or SH-malemide coupling are some exemplary non-limiting synthetic methods that may be used to modify a polysaccharide to form a drug delivery composition according to the disclosure. However, other covalent conjugation methods in light of this disclosure may be used.

Some exemplary polysaccharides that may be used to form the drug delivery compositions of the disclosure include chitosans, modified chitosans, a dextran modified to comprise a primary amine, other modified dextrans, glucosamines, hybrid polymers of any of the preceding polymers and/or any combinations thereof.

In some embodiments, a polysaccharide comprised in a drug delivery composition of the disclosure may be further capable of enduring rigors of a drug delivery vehicle synthesis and administration. A polysaccharide comprised in a drug delivery composition of the disclosure may be further able to associate or complex with a therapeutic nucleic acid or an anionic therapeutic agent.

Chitosan is a polysaccharide comprised of beta (1-4) linked units of glucosamine and N-acetyl glucosamine. Naturally occurring chitosan comprises primary amines and has various degrees of substitution with secondary amines in its N-acetyl glucosamine monomeric subunits and may be used for make drug delivery vehicles according to the disclosure.

Accordingly, in some embodiments, compositions of the disclosure may comprise a modified chitosan having at least one additional secondary amine. In some embodiments, a composition of the disclosure may comprise a modified chitosan having at least one additional tertiary amine. The additional amine molecules may be added by covalent conjugation or chemical conjugation (or other methods) of other secondary-amine carrying molecules and/or tertiary amine carrying molecules.

Exemplary secondary-amine and/or tertiary amine carrying molecules include imidazole-4-acetic acid, arginine, histidine, polyarginine, polyhistidine and/or combinations thereof.

For example, a composition according to the disclosure may include controlled conjugation of imidazole-4-acetic acid (IAA) to one or more primary amines of a polysaccharide (such as but not limited to chitosan) using carbodiimide chemistry. Other secondary and/or tertiary amines may be introduced onto a polysaccharide structure using similar carbodiimide chemistry methods.

In some embodiments, a chitosan may be modified to have a degree of substitution of about 10-15% secondary amines prior to addition of additional secondary amines and/or tertiary amines using the above described methods to obtain the compositions of the disclosure. In some embodiments, a chitosan (or any polysaccharide polymer) may comprise one or more primary, secondary as well as tertiary amines.

In some embodiments, the degree of substitution of a modified chitosan with at least one additional secondary amine and/or at least one tertiary amine is at least about 0.5% (as determined by NMR characterization methods described in Example 6 comprising evaluating imidazole peaks). In some embodiments, the degree of substitution of a modified chitosan having at least one additional secondary amine and/or at least one tertiary amine is at least about 1%, at least about 2% or at least about 3% (as determined by NMR characterization methods described in Example 6 comprising evaluating imidazole peaks).

In some embodiments, the degree of substitution of a modified chitosan with at least one additional secondary amine and/or at least one tertiary amine is at least about 10% (as determined by ninhydrin and NMR characterization methods described in Example 1). In some embodiments, the degree of substitution of a modified chitosan with at least one additional secondary amine and/or at least one tertiary amine is at least about 19.9% to at least about 30.2% (as determined by ninhydrin and NMR characterization methods described in Example 1).

In some embodiments, the degree of substitution of a modified chitosan with at least one additional secondary amine and/or at least one tertiary amine is at least about 0.5%, at least about 1%, at least about 2%, at least about 3%, at least about 4%, at least about 5%, about 10%, about 15%, about 16%, about 17%, about 18%, about 19%, about 19.5%, about 19.9%, about 20%, about 20.2%, about 20.4%, about 20.5%, about 20.7%, about 20.8%, about 20.9%, about 21%, about 22%, about 23%, about 25%, about 26%, about 27%, about 28%, about 29%, about 30%, about 30.2%, about 30.4%, about 30.6%, about 30.8%, about 31%, about 32%, about 35%, about 40%, about 45%, about 50%, about 55%, about 60%, about 65%, about 70%, about 75%, about 80%, about 85%, about 90%, about 95%, about 99% as may be determined by methods described in Example 1 or Example 6.

Chitosan has been used in the past as a non-viral, cationic carrier for gene delivery such as delivery of DNA vaccines via the oral route taking advantage of its mucoadhesive properties and as a carrier for siRNA. Chitosan has also been shown to carry nucleic acids for intranasal, intrabiliary, and intravenous administrations.

Previous characterization of some chitosan-pDNA nanoparticles showed nanoparticle formations of 150-250 nm with zeta potentials up to +18 mV. The characterization also showed limitations of solubility and buffering capacity of chitosan-pDNA in aqueous solution when compared to other non-viral gene delivery vectors such as polyethylenimine. An additional drawback of chitosan is that the inherent transfection efficacy of native chitosan is significantly less than other cationic polymers (e.g. PEI, dendrimers) or lipids. For example, previous in vitro studies have shown differences in protein expression by orders of magnitude when compared to PEI or lipofectamine based transfection. Lower transfection efficiencies may in part be due to low buffering capability and poor solubility of chitosan at neutral pH as well as incomplete dissociation of the nucleic acid in the cytoplasm.

Development of chitosan derivatives to enhance both in vitro and in vivo transfection has so far mostly focused on modification of its free primary amines. For example, trimethylation of chitosan has been shown to enhance solubility at physiological pH, while thiolation has been shown to enhance mucoadhesive properties. Polyethylene glycol conjugation to chitosan provides enhanced serum stability and reduced nanoparticle aggregation with no further reduction in transfection efficiency. Deoxycholic acid conjugation to chitosan has shown enhanced formation of self-aggregated nanoparticles with pDNA. Galactosylated chitosan-PEG conjugates have also shown enhanced targeting of nanoparticles with plasmid DNA to the liver. Polyethyleneimine (PEI) grafted to chitosan showed enhanced transfection of plasmid DNA with improved endosomal escape, however the cytotoxicity of PEI poses a significant concern in real life applications.

However, despite these efforts chitosan is limited as a pharmaceutical delivery vector because of poor solubility above pH 6.5, low buffering at endosomal and physiological pH (5.5 to 7.4), and poor cytoplasmic dissociation kinetics, resulting in in vitro transfection efficiencies that are magnitudes lower than other polymeric and lipid-based delivery vectors. Accordingly, all efforts in the art have failed to address the shortcomings of chitosan with regards to specific transport barriers, solubility, buffering capacity and/or improved intracellular nucleic acid delivery and none have provided efficient transfection.

In stark contrast to the art, chitosan-based drug delivery compositions of the present disclosure comprising at least one secondary amine or at least one tertiary amine and complexed with a therapeutic agent, when used as therapeutic nucleic acid or therapeutic anionic agent delivery vehicles have a high transfection efficiency.

For example, in some embodiments, an increase in the degree of substitution of a modified chitosan (i.e., increase in the number of secondary or tertiary amine residues on a chitosan polymer) may increase the transfection efficiency. Accordingly, an increased delivery of therapeutic agent to a target site in a subject or in vitro system may be achieved. A corresponding change in transcription or translation of one or more RNA or protein products encoded or controlled by a therapeutic nucleic acid that is complexed or associated with a modified chitosan of the disclosure may be obtained to achieve the desired therapeutic effect. In embodiments relating to an anionic therapeutic agent, more amount of such an agent may be efficiently delivered to a target site to mediate its therapeutic effect.

Compositions of the disclosure may also have a high effective buffering capacity in aqueous solutions. In some embodiments, buffering capacity past pH 8 may be achieved at about 20% degree of substitution (as analyzed by ninhydrin assay and NMR characterization methods described in Example 1) of chitosan with secondary and/or tertiary amine residues. In some embodiments, the compositions of the disclosure may have a buffering capacity in aqueous solutions of from about pH 4.5 to about pH 8.5.

In some embodiments, increased buffering may be achieved at higher degrees of substitution of chitosan with secondary and/or tertiary amine residues. For example, the buffering capacity of chitosan with a degree of substitution with an additional secondary amine and/or a tertiary amine of about 19.9% may be between about pH 6.5 and pH 8, while the buffering capacity of chitosan with a degree of substitution of about 30.2% may be between about pH 6.0 and pH 8.5 (degrees of substitution in this example are as determined by ninhydrin assay and NMR characterization methods described in Example 1).

In some embodiments, an increase in degree of substitution with secondary and/or tertiary amines may result in increased solubility of chitosan. In some embodiments, an increase in degree of substitution with secondary and/or tertiary amines may result in complete solubility of chitosan in an aqueous solution at pH of about 8. In some embodiments, where the degree of substitution of chitosan is about 20%, the modified chitosan may be at least 90% soluble at above pH 8 (degrees of substitution in this embodiment are as determined by ninhydrin assay and NMR characterization methods described in Example 1). In some embodiments, a modified polysaccharide according to the disclosure may be at least 90%, at least 95%, at least 99% or completely soluble in an aqueous solution at a pH greater than about 7, such as the physiological pH of about 7.4.

A chitosan based or other modified polysaccharide based drug delivery composition of the disclosure may further comprise one or more PEG molecules which may in some embodiments increase the serum stability.

Accordingly a drug delivery compositions of the disclosure comprising a modified chitosan or other modified polysaccharide as described herein may have or may provide one or more of the following advantages including: an increased solubility, increased stability, enhanced buffering capacity in an aqueous solution, enhanced endosomal escape properties, reduced or no biological toxicity, may facilitate cytoplasmic release of a complexed nucleic acid and/or an anionic therapeutic agent, may provide efficient transfection of a nucleic acid and/or may provide efficient delivery of an anionic therapeutic agent.

Therapeutic Agents

A therapeutic agent that may be delivered by the delivery compositions of the disclosure may include, but are not limited to, an anionic agent or a therapeutic nucleic acid.

A therapeutic anionic agent may include, but are not limited to agents such as an anionic protein, an anionic glycosaminoglycan, an anionic peptide, an anionic hormone, an anionic biomolecule or an anionic small molecule.

A therapeutic nucleic acid may include any nucleic acid such as but not limited to a polynucleotide, a DNA sequence, a DNA sequence encoding a therapeutic protein, an RNA sequence, a small interfering RNA (siRNA), a micro RNA (miRNA), an antisense oligonucleotide, a triplex DNA, a plasmid DNA (pDNA) or any combinations thereof

In some embodiments, a therapeutic nucleic acid may be treated or chemically modified. For example, a therapeutic nucleic acid may contain inter-nucleotide linkages other than phosphodiester bonds, such as phosphorothioate, methylphosphonate, methylphosphodiester, phosphorodithioate, phosphoramidate, phosphotriester, or phosphate ester linkages, which in some embodiments may confer increased stability. Nucleic acid stability may also be increased by incorporating 3′-deoxythymidine or 2′-substituted nucleotides (substituted with, e.g., an alkyl group) into the nucleic acid during synthesis or by providing the nucleic acid as phenylisourea derivatives, or by having other molecules, such as aminoacridine or poly-lysine, linked to the 3′ end of the nucleic acid. Modifications of a RNA and/or a DNA may be present throughout the oligonucleotide or in selected regions of the nucleic acid, e.g., the 5′ and/or 3′ ends, for example by methylation.

The discovery of RNA interference (RNAi), as an innate process in mammalian cells has pioneered the use of gene silencing as a new approach in treating a variety of diseases. Classical approaches to decreasing protein levels through gene silencing, such as oligodeoxynucleotides (ODNs), DNAzymes, ribozymes, or aptamer-based technologies have all shown some success, however, recent findings have demonstrated that synthetic small interfering RNAs (siRNAs), a component within the RNAi mechanism, provides highly efficient, specific and superior gene silencing within mammalian cells, thus prompting their extensive evaluation as potential therapeutic agent in numerous diseases.

However, like other nucleic acid-based therapies, siRNA-based approaches are hindered by serum instability and rapid degradation of the RNA molecules, poor intracellular penetration of naked siRNA and lack of inherent tropism. Therefore, only minimal success has been demonstrated with the use of naked siRNA in vivo, showing significant gene silencing only when directly administered to the tissue of interest at a very high dose which may however also lead to potential off-target silencing. Direct chemical modifications of siRNA have been reported for enhancing stability, cellular uptake, and targeting, however the majority of these chemical modifications only provide minimal improvement on overall delivery efficiency. To overcome these limitations, both viral and non-viral delivery vectors, are being extensively explored for enhanced delivery of siRNA.

Although viral vectors have proven to be highly efficient nucleic acid carriers, inherent safety concerns such as cytotoxicity, oncogenicity and immunogenicity limit their potential use. In vivo lipid-based delivery vectors for siRNA have demonstrated efficient delivery, however, in addition to systemic cytotoxicity concerns, most have been target-specific with predominant uptake in the liver, thus providing limited versatility. The lipid-based vectors are also limited due to potential serum instability, systemic toxicity, and accumulation in the liver.

These shortcomings lead to the development of polymer-based vectors, which provide the advantages of tailored biocompatibility, ease of modification, and low cost of production. Conjugation of peptides such as TAT(48-60) to siRNA has also shown the capability to improve gene silencing effects. In recent years, polymer-based carriers for siRNA have garnered significant attention because of their potential biocompatibility, modifiability, and ease of production. Specific focus has been placed on cationic polymers which form nanoscale polyplexes with siRNA, thus potentially minimizing serum degradation and allowing for endocytic intracellular uptake. However, biocompatible polymers are hindered by minimal in vivo efficiency.

Several modifications of chitosan have been tested previously for delivering siRNA molecules. However, as set forth above, all these prior modifications have failed due to their poor solubility above pH 6.5, low buffering at endosomal and physiological pH values (of about pH 5.5 to pH 7.4), and poor cytoplasmic dissociation kinetics, which have caused in vitro transfection efficiencies that are magnitudes lower than other polymeric and lipid-based delivery vectors.

The present disclosure provides polysaccharide based compositions as described here for efficient transfection and expression of therapeutic nucleic acids (e.g., pDNA, siRNA). Some example embodiments of the disclosure teach an imidazole-functionalized chitosan operable for pDNA and/or siRNA delivery.

Drug delivery compositions of the disclosure comprising an imidazole-functionalized chitosan demonstrated a significant increase in in vitro transgene expression as well as highly efficient in vitro gene silencing compared to non-modified chitosan (See, section entitled Example for details).

Enhanced transfection efficacy may be due to the introduction of secondary and tertiary amines to the chitosan polymer backbone which in some embodiments are shown to increase solubility at physiological pH without altering cyto-compatibility of the polymer.

A lipid-based and natural surfactant Infasurf has been shown to effectively silence GAPDH protein expression within the lungs up to 70% following intranasal administration at similar doses as described in the present Examples. While highly effective, Infasurf raises concerns due to potential side effects, which can include cyanosis and airway obstruction. Furthermore, high levels of GAPDH silencing in the heart and kidney may lead to undesired effects. Despite Infasurf's success, other liposomal delivery vectors, such as Genzyme Lipid 67 (GL67) have shown to be ineffective in providing significant siRNA-mediated silencing of target proteins following intranasal administration even at siRNA concentrations 8-fold greater than described in the present disclosure. The use of TransIT TKO, a cationic polymer/lipid combination, has also demonstrated significant enhancement of siRNA delivery and silencing effect following intranasal administration reducing target viral titer by up to 99%, however at siRNA doses 7-fold greater than shown in the present disclosure.

Synthetic Methods

The present disclosure also provides methods for synthesizing drug delivery vehicle compositions described herein. In one embodiment, a method for synthesis of the delivery vehicle compositions of the disclosure comprises providing a polysaccharide and a reactive compound; allowing the reactive compound to react with the polysaccharide so as to introduce at least one secondary amine or at least one tertiary amine onto the polysaccharide, thereby obtaining a modified polysaccharide comprising at least one secondary amine or at least one tertiary amine. The method may also comprise optionally adding at least one additional secondary amine to the composition. The method also comprises associating or complexing one or more therapeutic nucleic acids and/or one or more therapeutic anionic agents with a modified polysaccharide to obtain the delivery vehicle composition of the disclosure.

For example, in one embodiment, a polysaccharide of the disclosure, such as but not limited to, a chitosan, a modified chitosan, a dextran modified with primary amines, a glucosamine, a hybrid polymer of any of the preceding polymers or any combinations thereof may be reacted with a reactive compound comprising an imidazole, such as but not limited to, imidazole-4-acetic acid. The imidazole-4-acetic acid may react with a polysaccharide by carbodiimide chemistry to obtain compositions of the disclosure comprising a polysaccharide comprising at least one secondary amine and/or at least one tertiary amine.

In other embodiments, a polysaccharides of the disclosure may be reacted with a reactive compound such as, but not limited to, arginine, histidine, polyarginine, polyhistidine, or any other secondary and/or tertiary amine carrying molecules or combinations thereof, to obtain compositions of the disclosure comprising a polysaccharide comprising at least one secondary amine or at least one additional secondary amine and/or at least one tertiary amine.

In a method of the disclosure, according to some embodiments, the reacting step may continue until the degree of substitution of polysaccharide with a secondary amine, and/or an additional secondary amine; and/or a tertiary amine is about 0.5%, about 1%, about 2%, about 3%, about 4%, about 5%, about 6%, about 7%, about 8%, about 9%, about 10%, about 15%, about 16%, about 17%, about 18%, about 18.5%, about 19%, about 19.5%, about 19.9%, about 20%, about 21%, about 22%, about 25%, about 30%, about 31%, about 35%, about 40%, about 45%, about 50%, about 55%, about 60%, about 65%, about 70%, about 75%, about 80%, about 85%, about 90%, about 95%, about 99%. Degrees of substitution may be determined by either of the two NMR methods described in Examples 1 (ninhydrin assay and NMR) or Examples 6 (NMR comprising evaluating imidazole peaks).

In some embodiments, the controlled reaction produces compositions wherein the buffering capacity in an aqueous solution of a polysaccharide modified with a secondary amine and/or a tertiary amine is in the range of from about pH 4.5 to about pH 8.5.

In some embodiments, a method for producing a drug delivery composition of the disclosure may further comprise formulating a nanoparticle from the modified polysaccharide conjugated to a therapeutic agent (nucleic acid and/or anionic agent).

In some embodiments, a nanoparticle formulation of the disclosure may be further associated with polyethylene glycol (PEG). While not wishing to be bound to theory, association with PEG may prevent or reduce in vivo nanoparticle aggregation and enhance serum stability. Accordingly, PEGlyation of a nanoparticle of the disclosure may provide a formulation for enhanced systemic (e.g. intravenous) delivery.

A method of producing a drug delivery vehicle composition of the disclosure may therefore further comprise the steps of mixing and incubating nanoparticles with poly(ethylene glycol) succinimidyl valerate (mPEG-SVA). In one embodiment the molecular weight of PEG is Mw=5,000. However, PEG of different molecular weights may be used.

In some embodiments, a method of the disclosure for producing drug delivery vehicles operable for enhanced delivery of a therapeutic nucleic acid and/or an anionic agent may have one or more of the following advantages including: providing a drug delivery vehicle with increased solubility, increased in vivo stability, decreased nanoparticle aggregation, providing a drug delivery vehicle with enhanced buffering capacity in an aqueous solution, providing a drug delivery vehicle with enhanced endosomal escape properties, providing a drug delivery vehicle with reduced or no biological toxicity, providing a drug delivery vehicle that may facilitate cytoplasmic release of a complexed nucleic acid or an anionic therapeutic agent, providing a drug delivery vehicle that may provide efficient transfection of a nucleic acid, providing a drug delivery vehicle that may provide efficient delivery of an anionic therapeutic agent.

Therapeutic Methods

The present disclosure also provides methods for delivering and/or administering therapeutic nucleic acids or therapeutic anionic agents to a subject or patient in need thereof. A therapeutic method of the disclosure may comprise: 1) formulating a drug delivery vehicle comprising: a) providing a polysaccharide, and a reactive compound; b) allowing the reactive compound to react with the polysaccharide so as to introduce at least one secondary amine or at least one tertiary amine onto the polysaccharide to create a modified polysaccharide; c) optionally introducing at least one additional secondary amine on the modified polysaccharide; d) providing a therapeutic nucleic acid sequence and/or a therapeutic anionic agent; and e) associating or complexing the nucleic acid sequence and/or the anionic therapeutic agent to the modified polysaccharide to create a drug delivery vehicle, and 2) administering the drug delivery vehicle to the patient.

A therapeutic method of the disclosure may also comprise: 1) obtaining a drug delivery vehicle comprising: a) a modified polysaccharide having at least one secondary amine or at least one tertiary amine; and b) a therapeutic nucleic acid sequence and/or a therapeutic anionic agent; and 2) administering the drug delivery vehicle to the patient. In some embodiments, the drug delivery composition may be a nanoparticle formulation.

The disclosure also describes preparing and administering pharmaceutically acceptable drug delivery vehicles suitable for administering to a human patient in need thereof.

In some example embodiments, the present disclosure provides an in vivo evaluation of drug delivery compositions of the disclosure (e.g. imidazole-modified chitosan (chitosan-IAA) drug delivery compositions conjugated to siRNA or to pDNA). The ability of the present drug delivery compositions to function as a delivery vectors for siRNA to tissues such as liver and lungs following administration of the compositions by multiple routes of administration have been evaluated in example embodiments described later in the section entitled Examples.

Chitosan-IAA based drug delivery compositions of the disclosure were found to be highly efficient in delivering bioactive siRNA in vivo via intravenous and intranasal routes of administration. Intravenous administration with PEGylated siRNA-chitosan-IAA nanoparticles resulted in up to 50% average gene silencing in the lungs and up to 40% silencing in the liver in a dose dependent manner, with significant gene silencing even at a low 1 mg/kg dose. Intranasal administrations of non-PEGylated nanoparticles lead to, on average, 50% silencing of the target gene in the lungs. Additional details regarding the testing and results may be found in the sections entitles Examples.

In some embodiments, the disclosure comprises methods for treatment and/or prevention and/or alleviation of certain pathological or physiological disease conditions wherein a therapeutic nucleic acid or an anionic agent may be used. Such diseases and conditions may include any disease that may be treated or alleviated by a therapeutic nucleic acid and/or an anionic therapeutic agent. Some non-limiting examples of such diseases include cancers, tumors, HIV, diabetes, genetic disorders, neurodegenerative diseases, inflammatory disorders, heart disease (e.g. cholesterol reduction), and bacterial, viral or fungal infections.

For example, Apolipoprotein B is the predominant carrier protein for low-density lipoproteins (LDL) within the blood, which is responsible for the transport of cholesterol throughout the tissues. Recent studies have demonstrated the therapeutic potential of siRNA in silencing ApoB for the treatment and prevention of coronary artery disease. Intravenous delivery of cholesterol-conjugated anti-ApoB siRNA and the resultant silencing of the gene have been shown to lower total cholesterol. The potential of such treatments, however, is limited due to the high doses (≧20 mg/kg) necessary to achieve desired effects. The present disclosure investigated the efficacy of chitosan-IAA in delivering anti-ApoB siRNA to the liver in experiments that are described ahead in the section entitled Examples. Mice were treated intravenously with drug delivery vehicle compositions in accordance with this disclosure comprising PEGylated chitosan-IAA/siRNA nanoparticles. About (˜13%) silencing of the ApoB gene expression was noted in the whole liver at 1 mg/kg siRNA dose and significant knockdown (˜37%) was seen at the 3 mg/kg dose. Sample variability was noted with several mice demonstrating up to ˜50%-70% knockdown of ApoB. This may in-part be due to inherent variability of ApoB levels in mice as well as on dependence on dietary intake of each mouse. However, the present compositions demonstrate successful delivery and knockdown of target gene ApoB. Accordingly, therapeutic methods of the disclosure may be used to treat or reduce incidence of heart disease for example by reducing cholesterol.

In some embodiments, a therapeutic method of the disclosure may ameliorate or reduce the potential for developing heart disease in a subject by reducing cholesterol and may comprise: administering to the subject a pharmaceutical composition comprising: a) a modified polysaccharide (e.g., chitosan-IAA) of the disclosure having at least one secondary amine or at least one tertiary amine; and b) an anti-ApoB siRNA. In some embodiments, the composition comprising chitosan-IAA and anti-ApoB siRNA may be a nanoparticle formulation and may optionally comprise PEG. The Examples ahead describe an animal model study describing these embodiments.

Pharmaceutical Formulations and Delivery

Drug delivery compositions of the present disclosure may be used in the manufacture of medicaments and for the treatment of humans and other animal subjects or patients by administration in accordance with conventional procedures, such as an active ingredient in pharmaceutical compositions. While it is possible for a drug delivery vehicle of the disclosure to be administered alone, it may be preferable to present it as a pharmaceutical formulation comprising at least one active ingredient (e.g., an amine-modified polysaccharide vehicle comprising a therapeutic nucleic acid/or a therapeutic anionic agent) together with one or more pharmaceutically acceptable carriers therefore and optionally other therapeutic agents.

According to some embodiments, a carrier may be acceptable in the sense of being compatible with the other ingredients of the formulation and not injurious to the mammal. Pharmaceutical compositions of drug delivery vehicles of the disclosure may comprise combinations of polysaccharides of the disclosure with a therapeutic nucleic acid or therapeutic anionic agent and a carrier, inert or active, making the composition suitable for diagnostic or therapeutic use in vitro, in vivo or ex vivo.

A pharmaceutically acceptable carrier may encompasses any of the standard pharmaceutical carriers, such as a phosphate buffered saline solution, water, and emulsions, such as an oil/water or water/oil emulsion, and various types of wetting agents. The compositions also may include stabilizers and preservatives. For examples of carriers, stabilizers and adjuvants, see Martin, REMINGTON's PHARM. SCI., 15th Ed. (Mack Publ. Co., Easton (1975)), incorporated by reference herein. An effective amount may be an amount sufficient to effect beneficial or desired results. An effective amount may be administered in one or more administrations, applications or dosages. The pharmaceutical compositions of the invention may be administered to a mammal such as a human patient in need thereof. Mammals may include, but are not limited to, humans, murines, simians, farm animals, sport animals, and pets.

In some embodiments, the disclosure provides methods for preparing pharmaceutical formulations compatible for systemic delivery. For example, while intravenous administration provides rapid delivery of therapeutic molecules throughout the body, but toxicity due to particle aggregation, serum incompatibility as well as renal and hepatic clearance, limit the potential of delivery vectors for use through this route. A major challenge in nanoparticle-mediated siRNA delivery is the inherent “dilute” nature of the formulations that limits in vivo injection doses, especially for systemic delivery. Yet another problem in the art is the inability so far to concentrate siRNA nanoparticle formulations without particle aggregation.

The present disclosure provides methods to successfully concentrate nanoparticle formulations of its polysaccharide based drug delivery vehicles (e.g. chitosan-IAA/siRNA nanoparticles) at various doses as reported in the section ahead entitled Examples without significant aggregation of the nanoparticles.

The present disclosure, in some embodiments also provides methods to increase serum stability of siRNA nanoparticles (therapeutic nucleic acids) for intravenous administration comprising conjugating PEG groups the surface of the nanoparticles of the disclosure comprising a therapeutic siRNA. For example, as reported in the section ahead entitled Examples, PEGylation increased the effective hydrodynamic diameter of the nanoparticles (from an average of ˜170 nm to 230 nm) while decreasing zeta potential (from +41.1 mV to +19.1 mV).

In some example embodiments, PEG having a molecular weight 5000 was evaluated to prevent particle aggregation and increase circulation time. However, various PEG sizes may be evaluated in accordance to the present teachings since PEG size directly affects the nanoparticle diameter. Accordingly, embodiments of the disclosure provide methods to improve bio-distribution and gene silencing efficacy based on changing nanoparticle diameters based on the molecular weight of PEG. For example, use of a PEG 2000 may provide a smaller effective diameter for a drug delivery nanoparticle, which may reduce uptake in liver and increase uptake into the lungs and other organs. The present disclosure in some embodiments also encompasses the use of smaller PEG-spacers in conjunction with the present drug delivery vehicles to reduced circulation time of nanoparticles comprising a therapeutic agent.

In some embodiments, the disclosure describes the introduction of targeting moieties on the drug delivery formulations of the disclosure to provide organ specific uptake. For example a nanoparticle formulation of the disclosure may have added on histological and/or cell-specific markers that cause binding of a nanoparticle having such a marker to a specific cell or tissue type. Exemplary markers may include cancer markers, tissue specific cell membrane proteins and the like.

Methods of Administration

Administration or delivery of a pharmaceutical composition of the present disclosure may comprise any method which ultimately provides the drug delivery vehicle of the disclosure comprising a therapeutic nucleic acid and/or anionic therapeutic agent to cell/tissue/organ or site it is needed at. The drug delivery vehicles of the present disclosure may be delivered to a patient by a variety of means, including, but not limited to, oral ingestion, sublingual administration, intranasal, intramuscular injection, subcutaneous injection, parenteral administration, intrabiliary or topical application.

In some embodiments parenteral administration may comprise intravenous administration, intraperitoneal administration, subcutaneous administration, intrathecal administration, injection to the spinal cord, intramuscular administration, intraarticular administration, portal vein injection, or intratumoral administration.

Topical administration may include administration to skin, eye, or any mucosal membranes.

In some embodiments, a pharmaceutical composition of the disclosure may be contacted with a target tissue by direct application of the composition to the tissue.

The drug delivery vehicles of the disclosure, comprising the amine modified polysaccharide carrying a therapeutic nucleic acid agent and/or a therapeutic anionic agent, may be introduced into a patient in an amount sufficient to produce a desired clinical effect, including, but not limited to, a change in gene expression (e.g., increase in expression, decrease in expression or silencing of a gene), a change in gene transcription, a change in translation, a change in protein structure, a post-translational modification of a protein, or the treatment and/or prevention and/or alleviation of one or more symptoms or a medical condition. In some embodiments, a change in gene expression may involve gene silencing.

Administration in vivo may be effected in one dose, continuously or intermittently throughout the course of treatment. During the initial determination of dosage requirements, monitoring parameters that define the condition/disease may be advisable. Methods of determining the most effective means and dosage of administration are known to those of skill in the art, in light of this disclosure, and may vary with the composition used for therapy, the purpose of the therapy, the target cell being treated, and the mammal being treated. Single or multiple administrations may be carried out with the dose level and pattern being selected by the treating physician. Some example multiple dosage regimens are described below with regard to delivery of siRNA nanoparticle formulations described here. Suitable dosage formulations and methods of administering the pharmaceutical compositions may be empirically determined by those of skill in the art in light of this disclosure.

In some embodiments, the disclosure related to administration of therapeutic nucleic acids such as siRNAs. While localized routes of delivery offer an effective method in organ specific therapies, there is a need for systemic delivery for siRNA-based treatment of numerous diseases and disorders. Intravenous administration provides rapid delivery of therapeutic molecules throughout the body, but toxicity due to particle aggregation, serum incompatibility as well as renal and hepatic clearance, limit the potential of delivery vectors for use through this route. As described earlier, one major challenge in nanoparticle-mediated siRNA delivery is the inherent “dilute” nature of the formulations that limits in vivo injection doses, especially for systemic delivery. Yet another problem in the art is the inability so far to concentrate siRNA nanoparticle formulations without particle aggregation.

The present disclosure provides methods to successfully concentrate nanoparticle formulations of its polysaccharide based drug delivery vehicles (e.g. chitosan-IAA/siRNA nanoparticles) at various doses as described in other sections of this specification without significant aggregation of the nanoparticles. The present disclosure, in some embodiments also provides methods to increase serum stability of siRNA nanoparticles (therapeutic nucleic acids) for intravenous administration comprising conjugating PEG groups the surface of the nanoparticles of the disclosure comprising a therapeutic siRNA.

Accordingly, nanoparticle toxicity due to aggregation following systemic administration may also be modified by PEGylation as described herein. For example, chitosan-IAA compositions of the disclosure have been shown to be effective systemic carriers for siRNA. However, at high doses there might be an increase in viscosity following particle concentration that may cause nanoparticle aggregation, especially at low PEG modification efficiencies and for lower Mw PEGs. Accordingly, in some embodiments therapeutic methods of the present disclosure may comprise optimal administration of drug delivery nanoparticle formulations by multiple administrations at lower doses of the nanoparticles (e.g., 1 to 2 mg/kg siRNA) over several days through the intravenous route rather than a large single dose.

As described in further detail in the section ahead entitled Examples, gene silencing efficacy following systemic administration of drug delivery compositions comprising a therapeutic nucleic acid (siRNA) is demonstrate in an animal model wherein naïve BALB/C or C57BL/6J mice were injected via the tail vein (intravenous) with anti-GAPDH or anti-ApoB siRNA. Lungs and liver were chosen as initial target organs since these organs are afflicted by several diseases and provide ideal targets for therapeutic use of siRNA. Following administration of PEGylated chitosan-IAA nanoparticles (an example drug delivery vehicle of the disclosure), significant gene silencing was achieved in the whole lungs and liver (˜50% and ˜30% reduction in average GAPDH protein levels) even at a low dose (1 mg/kg siRNA). In comparison, minimal or highly variable knockdown was observed in mice treated with non-modified chitosan nanoparticles.

The present disclosure also describes methods for administration of therapeutic agents comprised in the present delivery vehicles intranasally. Intranasal administration may provide a more direct delivery route to the lungs and upper airways, while effectively circumventing hepatic first-pass clearance. In some embodiments, the choice of intranasal route in conjunction with the mucoadhesive properties of chitosan and its derivatives provide a localized treatment with minimal potential systemic side effects. The present disclosure describes the use of drug delivery compositions comprising chitosan-IAA for intranasal delivery of siRNA in animal model experiments in the section entitled Examples. Briefly, naïve C57BL/6J or BALB/C mice were intranasally administered nanoparticle formulations at low siRNA doses. Effective gene silencing (up to 30%) was observed in the entire lungs. Furthermore, treatment of mice with multiple low doses (0.5 mg/kg) over three consecutive days provided significant silencing (˜52%) at a lower total dose than previously demonstrated with non-modified chitosan/siRNA nanoparticles.

### Examples

The following additional examples are offered to illustrate some embodiments of the invention, and should not be viewed as limiting the scope of the invention.

### Example 1

### Drug Delivery Compositions: Synthesis and Characterization Materials

Protasan UP CL113 (Chitosan chloride salt) was purchased from Novamatrix, Norway (MW=130,000 Da, Degree of Deacetylation=86%). Imidazole-4-acetic acid monohydrochloride was purchased from AlfaAesar, Ward Hill, Mass. EDC (1-ethyl-3-(3-dimethylaminopropyl)carbodiimide hydrochloride) and Snakeskin pleated dialysis tubing (3,500 MW cut-off) were purchased from Pierce Biotechnology, Inc., Rockford, Ill. Exgen500 was purchased from Fermentas, Hanover, Md. Ninhydrin reagent was from Sigma Aldrich, St. Louis, Mo. Plasmid pgWiz Luciferase was purchased from Aldevron, LLC., ND. Silencer GAPDH siRNA (a gift) and SiPORT Amine were obtained from Ambion, Inc., Austin, Tex. HEK293T cells were obtained from the American Type Culture Collection, Manassas, Va. Gibco modified DMEM was used for cell culture medium with all remaining cell culture reagents purchased through Invitrogen, Carlsbad, Calif.

Synthesis of Modified Chitosan

Imidazole-4-acetic acid monohydrochloride conjugation to the primary amines of chitosan was achieved via use of carbodiimide chemistry. The reaction was performed at various ratios of IAA to the number of primary amines in chitosan. The general synthesis was as follows: A solution of 0.5% w/v chitosan PCL113 (Protasan) was prepared by dissolving the PCL113 in 0.1 M MES buffer, pH 5. An imidazole acetic acid (IAA) solution (2.0% w/v in 0.1 M MES buffer, pH 5.0) was also prepared. Both solutions were held on ice, and variable amounts of IAA solution were then added to the chitosan solution to achieve various degrees of primary amine modification. The chitosan-IAA solution was added to a 20 M excess of EDC (in relation to IAA) to promote addition of IAA to the chitosan backbone and immediately vortexed for 60 seconds. The final solution was then left to react overnight with end over end mixing. Dialysis was performed using the Snakeskin pleated dialysis tubing for 24 hours with the following cycles: 3 times for 2 hours against 3 mM HCl buffer followed by 3 cycles of 6 hours in length against de-ionized water. Samples were then left to lyophilize for 24 hours.

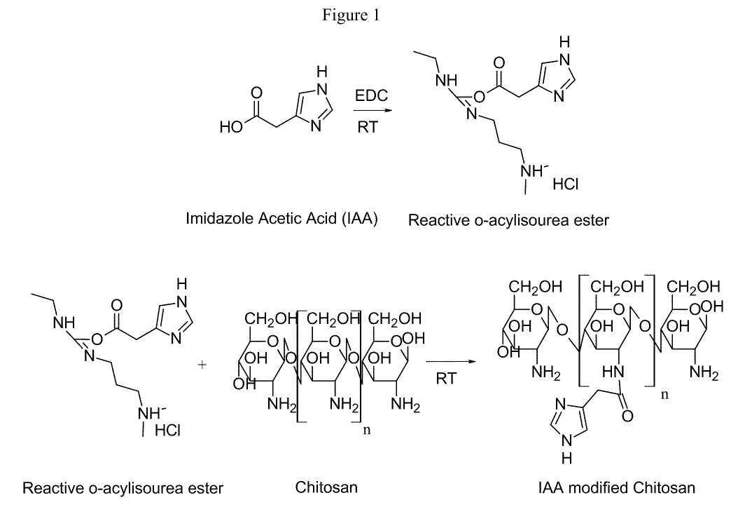

In a controlled manner, imidazole-4-acetic acid (IAA) was conjugated to the primary amines of chitosan using carbodiimide chemistry thereby introducing secondary and/or tertiary amines to the polysaccharide structure. A schematic of the conjugation scheme is shown in FIG. 1. EDC (1-Ethyl-3-[3-dimethylaminopropyl]carbodiimide hydrochloride) reacts with the carboxylic acid of imidazole acetic acid and forms an intermediate that is amine-reactive. This intermediate then reacts with the primary amines of chitosan forming an amide bond. The resulting polymer is a polysaccharide carrying primary, secondary as well as tertiary amines.

Ninhydrin Assay for Quantification of Degree of Modification

A ninhydrin assay was performed to determine the degree of IAA conjugation to the primary amines of chitosan. Briefly, a 0.2% w/v solution of chitosan was prepared by dissolving the PCL113 in 25 mM sodium acetate buffer, pH 5.5, and serially diluted to prepare the standards. A 0.05% w/v solution of each modified chitosan sample was prepared in the same sodium acetate buffer. The ninhydrin reagent was added to each solution in a 2:1 ratio and placed in a boiling water bath for 15-20 minutes. The optical density was then measured at 570 nm.

About 19.9% to 30.2% of chitosan primary amines were successfully modified with IAA (therefore the degree of substitution of chitosan with IAA was between 19.9% and 30.3%). Higher degrees of substitution (75% and 90%) were also achieved with increasing amounts of IAA. However, in order to preserve enough primary amines for nucleic acid condensation and for further ligand attachments to nanoparticles, the chitosan polymers with degrees of substitution of about 19.9% and about 30.2% were used for further analysis. The efficiency of the reaction ranged from about 60% to about 80% of the targeted modification.

¹H NMR Characterization

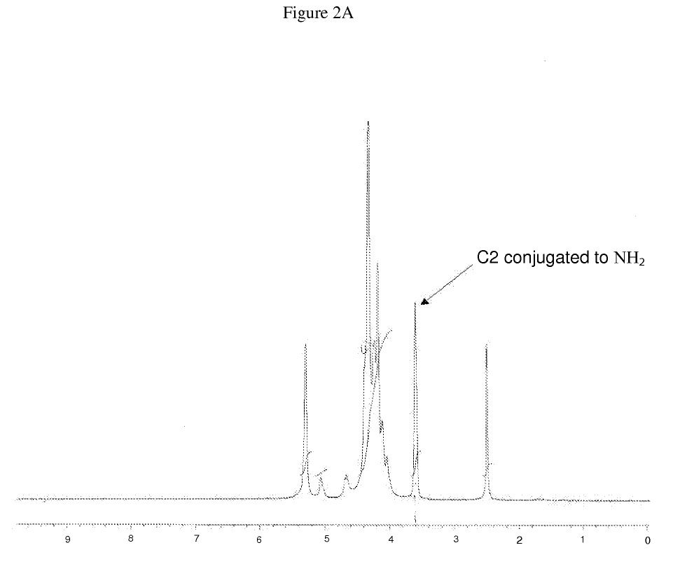

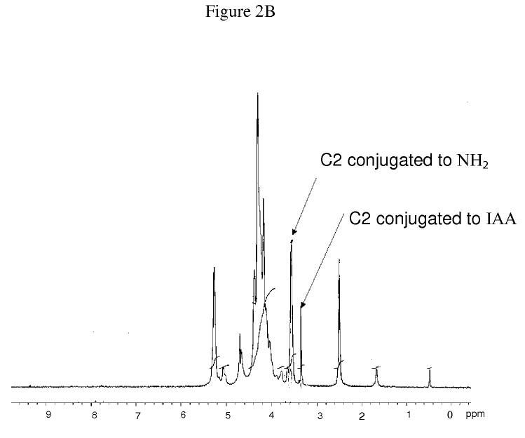

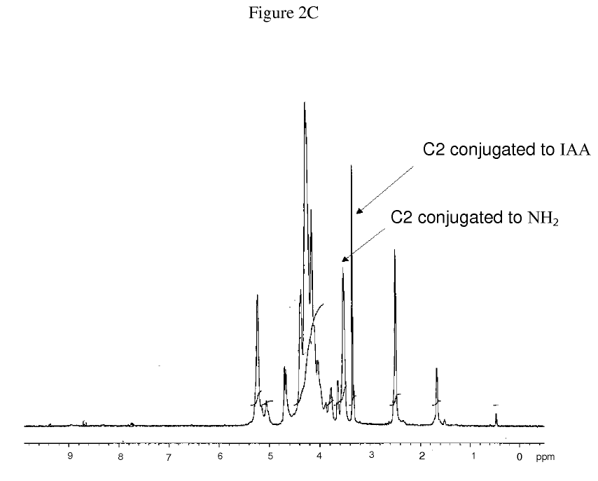

¹H NMR spectra of the experimental compounds were obtained with a Varian INOV-500 (500 MHz) at 80° C. in D₂O and spectra were displayed in PPM. As shown in FIGS. 2A, 2B and 2C show the NMR spectra for unmodified chitosan (FIG. 2A), chitosan-IAA 19.9% (chitosan with a 19.9% degree of substitution) (FIG. 2B), and chitosan-IAA 30.2% (chitosan with a 30.2% degree of substitution) (FIG. 2C). The NMR spectra indicates several characteristic peaks for chitosan including peak for the acetyl groups at 2.5 ppm, for C2 carbon at 3.6 ppm, and peaks for C1, C3, C4, C5, and C6 ranging from 4.2 ppm to 5.4 ppm. The C2 peaks in the modified chitosan samples show peak separation as a result of imidazole acetic acid addition to the primary amine resulting in a change of the harmonic frequency for the C2 carbons. This NMR signature increases with increasing degree of modification of the polysaccharide. As shown, going from a 19.9% degree of substitution to a 30.2% degree of substitution, the peaks from 3.2-3.4 ppm increases in size while the peak at 3.6 ppm reduces in size. As calculated from the ¹H NMR spectra, the degree of substitution for the two samples were 18.9% and 32.7% respectively.

Another method of NMR analysis was employed for some example embodiments of the disclosure including the in vivo experiments that are described in Examples 6-8. That method of NMR characterization provided quantification of degree of substitution through the evaluation of imidazole peaks (˜7.2 to 7.8 ppm and ˜8.0 to 8.8 ppm) and is described in Example 6. Degrees of substitution for similar polymers as measured by the NMR method using imidazole peak evaluation are ˜2-3% as compared to ˜30% as measured by the ninhydrin assay and NMR methods described in the present Example 1.

Buffering Capacity of Modified Chitosan

Effective transfection of nucleic acids may depend on the efficiency of endosomal escape of the nanoparticle carriers. Endosomal escape properties have been associated to buffering capacity of the carrier polymer in aqueous solution within the pH ranges of 5.2 to 7 by a mechanism referred to as “proton sponge.” Tests of the buffering capacity of the modified polysaccharides and imidazole acetic acids in aqueous solution were performed following the method described by Tang and Szoka (1997). Each sample was put into solution in 150 mM NaCl to a concentration of 0.5 mg/mL. Solutions were then titrated incrementally with 0.01N NaOH from pH below 4.0 to pH 9.0 with pH read after each increment.

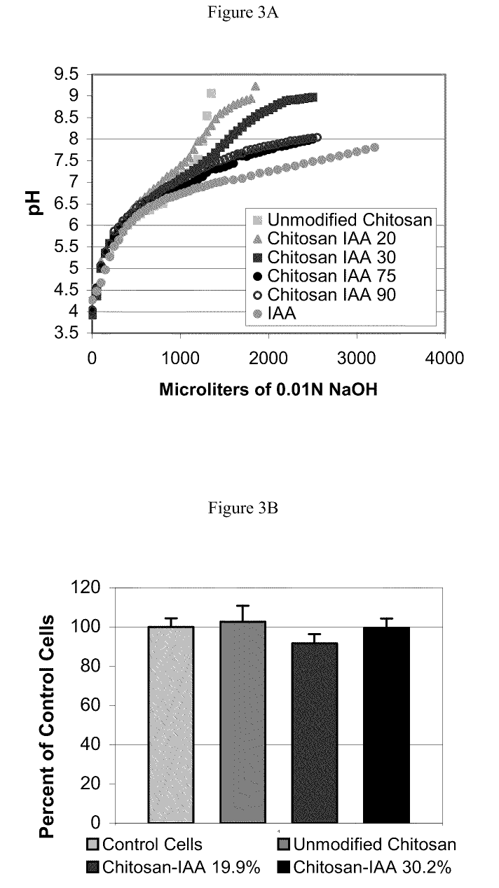

FIG. 3A depicts the buffering capacity of modified chitosan as compared to unmodified chitosan and IAA. As seen in FIG. 3A, limited buffering is achieved with unmodified chitosan, which may be due to the presence of primary amine alone as well as reduced solubility beyond pH 6.5. Imidazole acetic acid alone, although similar in buffering capacity from pH 5 to 6.5, demonstrated effective buffering at pH greater than 8 (data not shown). Chitosan-IAA was studied at various degrees of substitution (for example, 19.9%, 30.2%, 75%, and 90%). Effective buffering capacity was achieved past pH 8 with degrees of substitution of 19.9% and greater, with increased buffering at higher degrees of substitution. In some embodiments, increase in secondary and tertiary amine content allowed complete solubility of chitosan at pH>7.

The present disclosure demonstrates effective enhancement of in vitro transfection efficiency with a chitosan-based delivery vector in which several primary amines of the polysaccharide were functionalized with an imidazole-containing molecule (imidazole acetic acid), thereby introducing secondary and tertiary amine groups to the chitosan polymer backbone. The present disclosure also demonstrates enhanced buffering, increased solubility and minimal cytotoxicity of the chitosan-IAA derivative at both endosomal and physiological pH levels.

### Example 2

### Determination of Cytotoxicity of the Drug Delivery Compositions

MTT Assay

HEK293T cells were seeded in 96-well plates at a density of 10,000 cells per well in 0.2 mL of cell growth medium. After 24 hr, the medium was removed and replaced with 0.1 mL of serum free medium containing chitosan (modified and unmodified) and grown for an additional 48 hr. Each sample of chitosan was added in concentrations of 0.25 mg/mL (10-fold of transfection concentration) and performed in triplicate. The metabolic activity of each well was determined relative to control wells using the MTT assay. After incubation, 100 μL of Hank's Balanced Salt solution and 20 μL of MTT solution (5 mg/mL in Hank's Balanced Salt solution) were added to each well. After 3 hr at 37° C., 120 μL of MTT solubilization solution was added to break up the formazan crystals. The optical density of each well was then measured at 570 nm.

Minimal to no cytotoxicity was observed for the chitosan polymers with degrees of substitution of 19.9% and 30.2% as compared to untreated cells (FIG. 3B). Cell viability remained similar to the unmodified chitosan thus demonstrating no change in polymer toxicity following introduction of secondary and tertiary amines.

### Example 3

### Nanoparticle Formulations

Nanoparticle Formation

Separate nanoparticle formulations were prepared for both pDNA (pgWiz Luciferase) and siRNA (Silencer GAPDH siRNA) with chitosan and chitosan-IAA at various nitrogen to phosphate (N/P) ratios. For pDNA nanoparticles, chitosan and chitosan-IAA solutions (0.01-0.06% in 25 mM sodium acetate buffer, pH 5.5) and a pDNA solution of 4 μg/mL in 25 mM sodium sulfate were preheated to 50-55° C. separately. An equal volume of chitosan solution was added drop-wise to a pDNA solution and vortexed for 20-30 seconds. The final volume of the mixture in each preparation was 200 μL. Nanoparticle preparation for siRNA was performed identically with RNAse free solutions, 200 mM sodium acetate, and with siRNA in RNAse free water rather than sodium sulfate. The nanoparticles were used for transfection, cytotoxicity studies, particle sizing, zeta potential, and gel retardation assays.

Nanoparticle Characterization

For particle-mediated delivery of nucleic acids (in which the particles form the drug delivery vehicle), appropriate ranges of size and surface charge may result in effective cellular uptake. In some embodiments, cationic particles as well as particles below 500 nm may be used to enhance endocytosis. Modifications of the polymer and nucleic acid ratio as reflected in the N/P ratio) may significantly influence both size and zeta potential of the resulting nanoparticles. Therefore, particle size distribution was determined by Dynamic Light Scattering with a ZetaPlus system (Brookhaven Instruments Corporation, Holtsville, N.Y.). Zeta potential was also analyzed using the ZetaPlus system. Nanoparticle solutions of 1 mg/mL concentration were diluted in 1 mM KCl, and then read in the ZetaPlus system. Ten readings were taken for each sample. Nanoparticle formulations were prepared at different N/P ratios to study the effects on size and zeta potential.

As shown in Table 1, particles formed with the modified chitosan varied in size based on the N/P ratio and degree of substitution. Particles formed with the modified chitosans and pDNA were significantly larger in size compared to particles formed using the unmodified polymer. In addition, the particle size decreased with increasing N/P ratio for a given polymer. However, no significant difference in size was observed between the two modified polymers at a given N/P ratio. For example, for unmodified chitosan/DNA nanocomplexes, average particle size ranged from 161-311 nm, while the chitosans with degrees of substitution of 19.9% and 30.2% produced particles with average sizes from 267-861 nm and 324-831 nm, respectively (at various N/P ratios). Similar results were demonstrated with siRNA, however with smaller effective diameters than with pDNA (Table 1). Zeta potential was also significantly affected by both the N/P ratio and degree of substitution (Table 2). Average zeta potentials for the unmodified chitosan/DNA complexes ranged from 0.11 to 12.07 mV and increased with increasing N/P ratio. This significantly increased for both the chitosan-IAA 19.9% (19.88-26.05 mV) and chitosan-IAA 30.2% (23.06-24.9 mV). A similar trend was observed with siRNA nanocomplexes. However, no significant difference in zeta potential was observed between the two different modified chitosans.

TABLE 1

| N/P Ratio | | | |
|---|---|---|---|
| 5 | 10 | 25 | 50 |

Effective Diameter (nm) of Nanoparticles with pDNA

| | | | | |
|---|---|---|---|---|
| Unmodified Chitosan | 311.9 ± 4.5 | 225.2 ± 0.8 | 219.9 ± 6.2 | 161.8 ± 30.4 |
| Chitosan-IAA 20 | 861.1 ± 75.2 | 687.8 ± 34.6 | 472.3 ± 7.9 | 267.3 ± 1.7 |
| Chitosan-IAA 30 | 831.4 ± 27.8 | 712.5 ± 55.9 | 511.2 ± 17 | 324.1 ± 11.8 |

Effective Diameter (nm) of Nanoparticles with siRNA

| | | | | |
|---|---|---|---|---|
| Unmodified Chitosan | 218.1 ± 14.8 | 137.8 ± 4.4 | 111.2 ± 19.2 | 73.4 ± 23.5 |
| Chitosan-IAA 20 | 242.1 ± 9.6 | 206.7 ± 9.3 | 128.8 ± 4.9 | 152.9 ± 13.9 |
| Chitosan-IAA 30 | 295.7 ± 8.7 | 176.1 ± 5.7 | 163.1 ± 4.7 | 156.2 ± 4.7 |

TABLE 2

| N/P Ratio | | | |
|---|---|---|---|
| 5 | 10 | 25 | 50 |

Zeta Potential (mV) of Nanoparticles with pDNA

| | | | | |
|---|---|---|---|---|
| Unmodified Chitosan | 0.11 ± 0.64 | 10.76 ± 2.36 | 11.90 ± 3.05 | 12.07 ± 1.12 |
| Chitosan-IAA 20 | 19.88 ± 1.06 | 21.02 ± 0.85 | 21.94 ± 2.5 | 26.05 ± 0.87 |
| Chitosan-IAA 30 | 23.06 ± 0.82 | 24.21 ± 0.79 | 24.35 ± 0.47 | 24.9 ± 1.38 |

Zeta Potential (mV) of Nanoparticles with siRNA

| | | | | |
|---|---|---|---|---|
| Unmodified Chitosan | 15.73 ± 4.10 | 17.63 ± 3.76 | 20.28 ± 1.82 | 34.07 ± 2.28 |
| Chitosan-IAA 20 | 29.85 ± 3.64 | 29.85 ± 3.30 | 32.45 ± 2.98 | 38.28 ± 2.92 |
| Chitosan-IAA 30 | 30.63 ± 0.15 | 34.66 ± 8.05 | 33.89 ± 4.49 | 37.58 ± 1.90 |

### Example 4

### Transfection of Drug Delivery Compositions

In Vitro Transfection of HEK293T Cells with Chitosan-DNA Nanoparticles

In vitro transfection efficiency was performed on HEK293T using the pgWiz Luciferase plasmid. HEK293T cells were seeded 24 hr prior to transfection in a 24-well plate at a density of 5×10⁴ cells per well in 1.0 mL of complete medium (DMEM containing 10% FBS, supplemented with 1% penicillin and streptomycin). Nanoparticles were prepared with the modified and unmodified chitosan with varying concentrations of chitosan and amounts of DNA. The three types of chitosan studied were Protasan UP CL113 (unmodified chitosan), chitosan-IAA 19.9%, and chitosan-IAA 30.2%. Exgen 500 was used as a positive control. The amount of nanoparticles equivalent to 1.0 μg of DNA was added to each well and incubated with the cells for 4 hr in 0.5 mL of serum-free medium. After 4 hours, 0.5 mL of DMEM medium (20% FBS 2% penicillin and streptomycin) was then added each well to bring total volume with 1 mL and cells were further incubated for 44 hr at 37° C. before being analyzed for transfection efficiency. As a positive control, transfection with PEI-DNA complexes (Exgen 500) was performed. All transfection experiments were performed in triplicate.

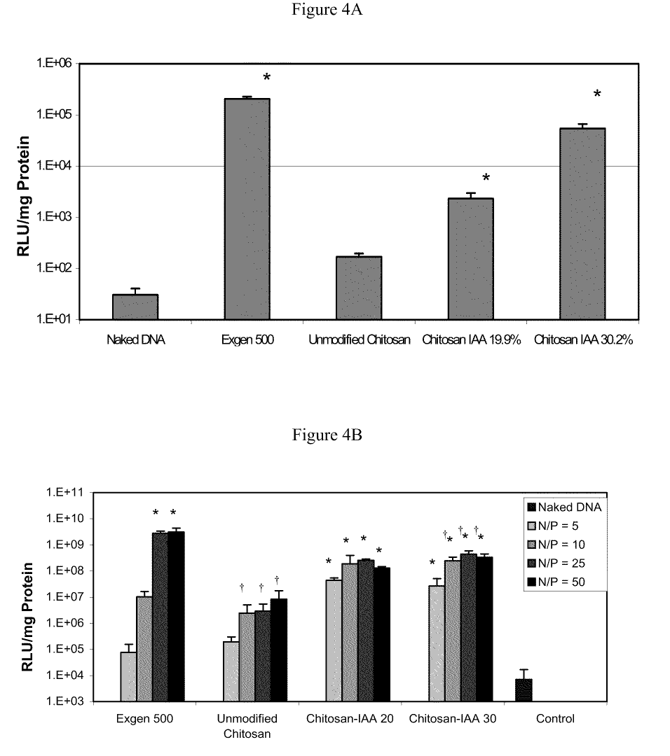

Transfection results (FIG. 4A) demonstrated a substantial increase in efficiency based on the degree of modification when equivalent amounts of chitosan or chitosan-IAA were used to prepare the nanoparticles. In FIG. 4A, the N/P ratios for unmodified chitosan, chitosan-IAA 19.9%, and chitosan-IAA 30.2% are 19, 24, and 26, respectively. All samples are n=3. *=p<0.05 as compared to unmodified chitosan at same N/P ratio or equivalent weight using t-test, †=p<0.05 as compared to unmodified chitosan at same N/P=5 sample within each chitosan polymer degree of modification using t-test. Greater than 10-fold increase in average luciferase expression was observed for the chitosan-IAA 19.9% and approximately 100-fold increase for the chitosan-IAA 30.2%.

When nanoparticles were prepared at various N/P ratios (FIG. 4B), some variations were seen in the transfection efficiencies. With an increase in N/P ratio, a significant increase in transfection was noted, for all polymers. However, cytotoxicity limitations were evident with Exgen 500 (PEI), which was used as a positive control for transfection efficacy. At lower N/P ratios (N/P=5, 10), both type of modified chitosans provided over 100-fold greater luciferase expression as compared to unmodified chitosan and Exgen 500 (a linear 25 kDa polyethyleneimine). The higher transfection efficiency compared to Exgen 500 is likely due to the higher number of protonated amine groups in the chitosans, whereas only 1 out of every 3 amines in Exgen 500 is protonated. At higher N/P ratios, the Exgen 500 was most effective; however cytotoxic effects of PEI, especially at high concentrations are well documented. The modified chitosans both showed greater than 10 to 100-fold higher efficiency than the unmodified chitosan, while retaining minimal cytotoxicity.

Luminometric Assay for Luciferase

Following transfection for 48 hours, the cells were permeabilized with 250 μL of Glo-Lysis buffer to release the luciferase protein for analysis. Luciferase activity in cell extracts was measured using a Bright-Glo luciferase assay (Promega, Madison, Wis.) on a luminometric plate reader. Normalization of RLU levels for each well was done by determination of overall protein content in each well using a Micro BCA protein assay kit (Pierce Biotechnologies, Inc., Rockford, Ill.).

### Example 5

### Drug Delivery Compositions for In Vitro Delivery of siRNA

In Vitro Transfection of HEK293T Cells with Chitosan-siRNA Particles

HEK293T cells were seeded 24 hr prior to transfection in a 96-well plate at a density of 1×10⁴ cells per well in 0.2 mL of complete medium (DMEM containing 10% FBS, supplemented with 1% penicillin and streptomycin). Nanoparticles formulations at various N/P ratios were studied. An amount of nanoparticles carrying 50 nM of siRNA was added to each well and incubated with the cells for 4 hr in 0.1 mL of serum-free medium. After 4 hours, 0.1 mL of DMEM medium (20% FBS 2% penicillin and streptomycin) was added to each well to bring total volume with 0.2 mL and cells were further incubated for 44 hr at 37° C. before being analyzed for transfection efficiency. As a positive control, transfection of the siRNA was performed using siPORT Amine. All transfection experiments were performed in triplicate.

Analysis of GAPDH Knockdown with Chitosan-IAA/siRNA Nanoparticles

GAPDH knockdown was analyzed using the KDalert GAPDH assay kit (Ambion, Inc., Austin, Tex.). After transfection for 48 hours, media was removed from each well and replaced with 0.2 mL of KDalert lysis buffer and incubated at 4° C. for 20 minutes. 10 μL of each lysate was then transferred to a new 96 well plate and 90 μL of KDalert master mix was then added to each well. Fluorescence levels for each well were then read immediately after addition of the master mix (λ_{Exc.}=560 nm and λ_{Emm}=590 nm). After 4 minutes, fluorescence of the plate was measured again, and the difference of the two readings was used to determine GAPDH activity.

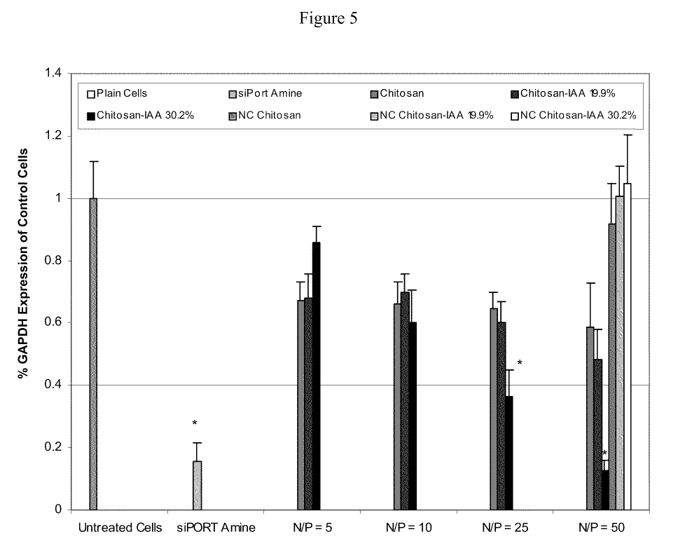

The results, shown in FIG. 5, demonstrate an increase in gene knockdown with increased N/P ratio for all three chitosan samples. Little gene silencing was observed at low N/P ratios (5 and 10) with any of the chitosan samples, however substantial increase, specifically with the chitosan-IAA 30.2% was seen at N/P ratios at or above 25. At N/P=25, the gene knockdowns for chitosan, chitosan-IAA 19.9%, and chitosan-IAA 30.2% were 35%, 40% and 64% respectively. These values increased to 41%, 52%, and 87% respectively at N/P=50. The 30.2% modified polymer matched the gene knockdown of the siPORT Amine positive control at N/P=50, while still well within the nontoxic dose range of the polymer. A negative control (NC) siRNA delivered by all three variations of the polymer at N/P=50 demonstrated no gene knockdown due to the polymer itself.

### Example 6

### Another Method for Synthesis of Imidazole-Modified Chitosan Materials

Chitosan (hydrochloride salt, Protasan UP CL113, Mw=130,000 Da, degree of deacetylation=86%) was purchased from Novamatrix, Norway. Imidazole-4-acetic acid monohydrochloride (IAA) and MES Hydrate were purchased from Acros Organics, Beel, Germany. EDC (1-ethyl-3-(3-dimethylaminopropyl)carbodiimide hydrochloride) and Snakeskin pleated dialysis tubing (10,000 MWCO) were both purchased from Pierce Biotechnology, Inc., Rockford, Ill. mPEG-Succinimidyl Valerate (mPEG-SVA, Mw=5,000 Da) was purchased from Laysan Bio, Arab, AL. Silencer® GAPDH siRNA, Silencer® Select ApoB siRNA, Silencer® Negative Control siRNA and siPORT Amine were provided by Ambion Products, Life Technologies Corporation, Austin, Tex.

Synthesis of Imidazole-Modified Chitosan

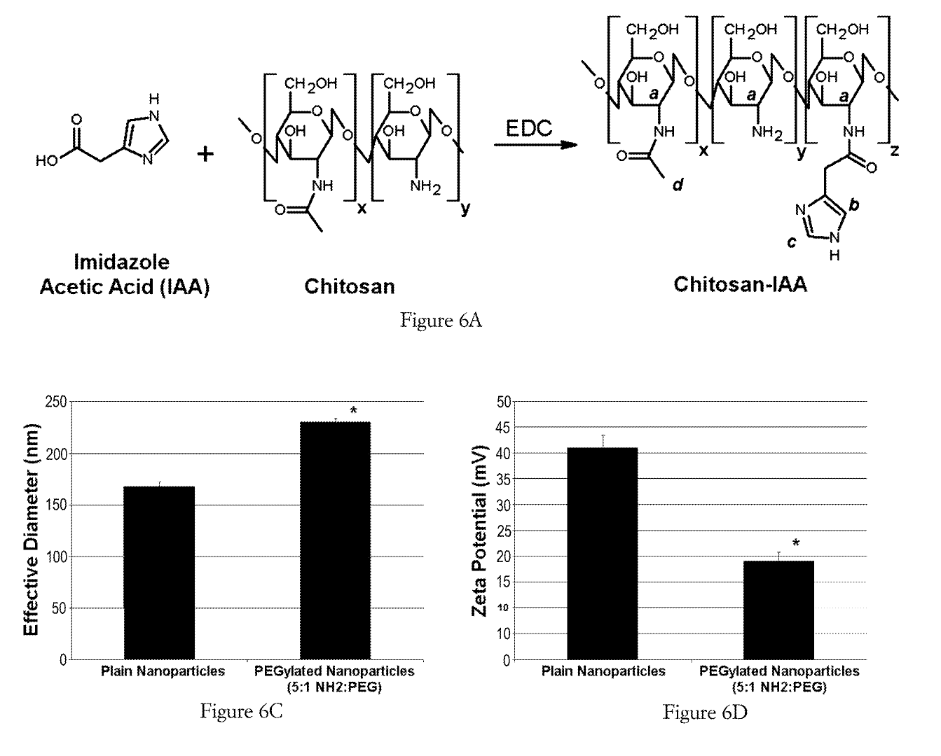

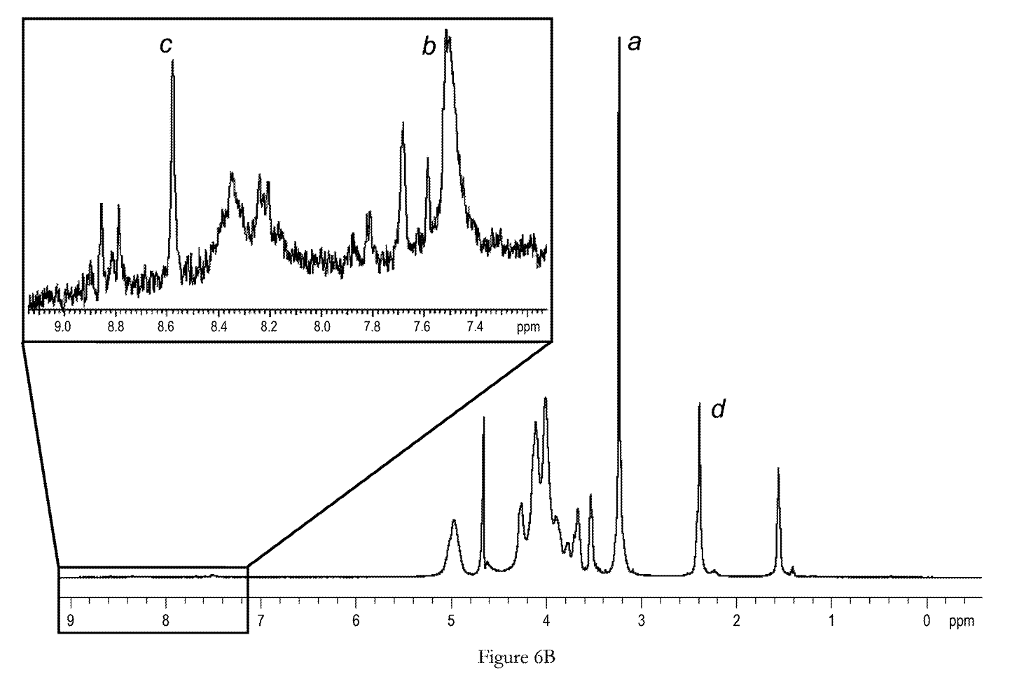

Conjugation of Imidazole-4-acetic acid monohydrochloride to the primary amines of chitosan was performed via carbodiimide chemistry as previously described in Example 1 with slight modification. Briefly, the ratio of IAA to the number of primary amines in chitosan was adjusted to provide variable degrees of modification. A schematic for this reaction and the final chemical structure of chitosan-IAA is shown in FIGS. 6A-6D. Chitosan (1% w/v, 49.3 mM NH₂) and imidazole acetic acid (2.0% w/v, 123 mM) solutions were prepared in 0.2M MES buffer (pH 5.5) and stored on ice. Chitosan (100 mg, 493 μmoles NH₂, 10 mL) was mixed with IAA (40 mg, 246.5 μmoles, ˜2.0 mL) and kept on ice. The combined solution was then used to dissolve 20M excess of EDC (945 mg, 4.93 mmole) to IAA and immediately vortexed for 60 seconds to catalyze conjugation of IAA along the chitosan backbone. The final solution was left to react overnight on an end-over-end rotator at room temperature. Reaction volumes were then dialyzed for 24 hours using Snakeskin pleated dialysis tubing against 5 mM HCl for 2 hours (3×), then against deionized water for 6 hours (3×). Samples were then lyophilized overnight. Degree of substitution was characterized via ¹H NMR with samples prepared in D₂O and measured at 70° C. using a Varian i.n.OV-500 (500 MHz) spectrometer with spectra shown in units of PPM.

Synthesis and Characterization of Imidazole-Modified Chitosan

IAA-modified chitosan (chitosan-IAA) was prepared using a one step carbodiimide reaction as described in Example 6. The chemical structure for this modified chitosan is shown in FIG. 6A. An improved method for quantification of the degree of substitution for the modified polysaccharides was determined via ¹H NMR by taking the ratio of the two imidazole proton peaks (˜7.2 to 7.8 ppm and ˜8.0 to 8.8 ppm), represented by “b” and “c” in FIG. 6B and the integration of the peaks correlating to the C2 protons of the IAA-N-acetyl glucosamine, N-acetyl-glucosamine and glucosamine subunits at ˜3.2 to 3.4 ppm (represented by “a” in FIG. 6B). Chitosan-IAA batches used for these studies were characterized to have an average of ˜2-3% degree of substitution. This is equivalent to approximately, on average, 15-23 molecules of IAA per chitosan polymer chain. IAA-modified chitosans demonstrated increased solubility and buffering as described earlier in this application (data not shown).

Nanoparticle Synthesis and Characterization

Nanoparticles were synthesized by complex coacervation of non-modified chitosan or Chitosan-IAA with siRNA. N/P ratios of 25 and 50 were used for particle synthesis. Non-modified nanoparticles ranged in size from 50 to 200 nm, and demonstrated a general trend of decreasing size with increasing N/P ratio (data not shown). PEGylated particles demonstrated a slightly higher size range (from 100 nm to 250 nm). Overall, the mean effective diameters for the non-modified and PEGylated nanoparticles, as shown in FIG. 6C, were 168 and 230 nm, respectively. As shown in FIG. 6D, reduction in zeta potential was also noted for particles following PEGylation. Surface charges for non-modified nanoparticles were +41.1 mV, while PEGylated nanoparticles demonstrated a surface charge of +19.1 mV.

### Example 7

### Nanoparticle Synthesis for In Vivo Administration

Nanoparticle Synthesis for Intranasal Administration

Nanoparticle formulations for intranasal administration were prepared with siRNAs (Silencer® Select GAPDH and Silencer® Negative Control) as previously described with slight modification. Intranasal nanoparticle synthesis was performed as follows: chitosan and chitosan-IAA solutions (0.01%-0.065% w/v) in RNase-free sodium acetate (200 mM, pH 4.5) were heated to 55° C. for 20 minutes. Solutions of siRNA (20 μg/mL) were prepared in RNase-free water and preheated to 55° C. for 1 minute prior to nanoparticle synthesis. Equivolumes of chitosan or chitosan-IAA solutions were added drop-wise to the siRNA solutions at various nitrogen:phospate (N/P) ratios (based on concentration of chitosan) while vortexing. Samples were then vortexed for 30 seconds resulting with final volumes of 200 μL and allowed to incubate at room temperature for 30 minutes. Following incubation, samples were combined and concentrated with Amicon Ultra centrifugal filtration units (Millipore, Billerica, Mass., MWCO=30,000) to provide doses of 0.5, 1, or 2.0 mg/kg (mg siRNA/kg body weight) in 20 μL volumes. Samples were centrifuged in filters at 1500×g for up to 40 minutes to adequately concentrate doses. Nanoparticles were then immediately characterized or used for in vivo studies. Nanoparticle size distribution was determined by Dynamic Light Scattering (DLS) using a ZetaPlus system (Brookhaven Instruments Corporation, Holtsville, N.Y.). Zeta potential (ζ) for the nanoparticles was also determined using the ZetaPlus system. Briefly, nanoparticle solutions in 1 mM KCl (pH5.5) were prepared at 0.25 mg/mL concentrations of polymer and read at room temperature with each sample read 10 times. Nitrogen to phosphate (N/P) ratio was determined as the ratio of protonable amines within the chitosan or chitosan-IAA at pH 4.5 to the phosphates of the siRNA.

Nanoparticle Synthesis and PEGylation for Intravenous Administration

For intravenously administered nanoparticles, synthesis was performed with 50 μg/mL siRNA solutions with chitosan and chitosan-IAA solutions adjusted accordingly. Particles were otherwise prepared identically to those described for intranasal administration at N/P ratios of 50 and 40, for GAPDH and ApoB studies, respectively, with the following modification. Following 20 minutes of incubation at room temperature, nanoparticles were surface modified with poly(ethylene glycol) succinimidyl valerate (mPEG-SVA, Mw=5,000) to prevent in vivo nanoparticle aggregation and enhance serum stability. Briefly, 10 mL of nanoparticles was mixed with mPEG-SVA (5% w/v in PBS, pH 7.4) and vortexed for 60 seconds. Samples were then incubated for 2 hours at room temperature in a molar ratio of 1:5 (PEG:NH₂ in chitosan or chitosan-IAA). Following incubation, samples were then concentrated in Amicon Ultra centrifugation filtration units to provide 200 μL volumes for each dose for in vivo studies. Nanoparticles were characterized as described for non-PEGylated nanoparticles.

### Example 8

### In Vivo Evaluation of Drug Delivery Compositions Mice

Female BALB/C and C57BL/6J mice for in vivo studies were purchased from Jackson Laboratory, Bar Harbor, Me. and housed at the Animal Resource Center, University of Texas at Austin. Mice were 6-13 weeks old during treatment with nanoparticles. All experiments were approved by the Institutional Animal Care and Use Committee (IACUC) at the University of Texas at Austin. Animals were provided care in accordance with procedures described in Animal Care and Use Handbook (University of Texas at Austin) and Principles of Laboratory Animal Care (NIH publication #85-23, revised in 1985).

Intranasal Administration of Chitosan-IAA Nanoparticles to C57BL/6J Mice

In vivo gene silencing of GAPDH, following intranasal administration of chitosan and chitosan-IAA/siRNA nanoparticles, was studied on naïve C57BL/6J or BALB/C mice. Mice were separated into groups for administration of various nanoparticle formulations to each group (n=5). PBS and Silencer® negative control siRNA were used as negative controls for each set of experiments. Mice were placed under anesthesia with isofluorane and were administered 20 μL (10 μL per nostril per mouse) of chitosan-siRNA, chitosan-IAA-siRNA, or siPORT Amine-SiRNA (positive control) nanoparticles. Doses were given either as a single dose of 0.5, 1.0, or 2.0 mg/kg siRNA per mouse or as three consecutive daily doses of 0.5 mg/kg siRNA/mouse/day (i.e. a total of 1.5 mg/kg of siRNA/mouse). Three days following final nanoparticle administration, mice were sacrificed and whole lungs harvested for analysis. Following immediate snap-freezing, tissues were manually homogenized and processed to determine GAPDH silencing as later described.

Intravenous Administration of PEGylated Nanoparticles to BALB/C and C57BL/6J Mice

Both BALB/C and C57BL/6J mice (ages ranging from 6-9 weeks) were studied for nanoparticle-mediated in vivo gene silencing of GAPDH and Apolipoprotein B (ApoB) following intravenous administration. In each experiment, mice (n=5 mice per group) were placed in a warm restrainer and intravenously administered with 200 μL of nanoparticle formulations (chitosan or chitosan-IAA) via tail vein injection at Silencer® Select siRNA doses of 1 mg/kg per mouse or 3 mg/kg per mouse (either a single dose (GAPDH) or two equal doses (ApoB)) over consecutive days. Prior to administration, nanoparticle solutions were thoroughly mixed to minimize potential aggregates. PBS and Silencer® negative control siRNA were used as negative controls for each experiment. Silencer Select siRNAs were prepared and developed with a novel algorithm and LNA modifications providing altogether improved potency and specificity as described in Elmen et al., 2005, “Locked nucleic acid (LNA) mediated improvements in siRNA stability and functionality,” Nucleic Acids Res., 33, 439-47. In all experiments, mice were treated with nanoparticles at either an N/P ratio of 40 (ApoB) or 50 (GAPDH). Three days following final injection, treated animals were sacrificed and organs (lungs and liver) were harvested and either snap-frozen and stored at −70° C. or placed into RNA later solution on ice and stored for further analysis. Tissue samples were then homogenized and analyzed for either GAPDH protein levels or ApoB mRNA levels as described in the following sections.

Analysis of GAPDH Protein Silencing in Tissue Samples

Following harvesting, organs were snap-frozen in liquid nitrogen and stored at −70° C. Tissues were then homogenized as previously described in Hanson et al., 1998, “Developmental changes in lung cGMP phosphodiesterase-5 activity, protein, and message,” Am J Respir Crit Care Med. 158, 279-88, and analyzed using the KDalert GAPDH assay kit (Ambion) according to manufacturer's protocol to determine protein levels of GAPDH. Briefly, homogenized tissue samples were resuspended in KDalert Lysis buffer (1 mL for lungs and 5 mL for liver) and incubated at 4° C. for 20 minutes. 10 μL of diluted lysate (1:1,000 to 1:10,000 dilutions) from each sample was then transferred to a fresh black 96 well plate and 90 μL of KDalert master mix was added to each well. Fluorescence levels for each well were read immediately after addition of the master mix (λ_{Exc.}=560 nm and λ_{Emm}=590 nm). Identical measurements were repeated after 4 minutes. GAPDH activity was determined by the difference of the two readings. Values were normalized to the total protein content (Micro BCA assay, Pierce Biotechnology) of the lysate sample.

RNA Isolation and qPCR Analysis of Gene Expression in Tissue Samples

To determine ApoB or GAPDH silencing, total RNA was isolated from tissue samples using mirVana™ PARIS™ Kit (Ambion) per manufacturer's protocol. Following total RNA isolation, samples were resuspended in nuclease free water, and treated for genomic DNA contamination with a Turbo DNA-free™ kit (Ambion). To ensure quality and determine the quantity of mRNA isolated, samples were analyzed on a ND-1000 spectrophotometer (Nanodrop, Wilmington, Del.). Complementary DNA was synthesized using a High Capacity cDNA Reverse Transcription Kit (Applied Biosystems, Foster City, Calif.). The reverse transcription was performed 25° C. for 10 minutes, 37° C. for 120 minutes, and completed at 85° C. for minutes. Samples were held at 4° C. cDNA samples were analyzed by quantitative polymerase chain reaction (qPCR) to determine gene expression level using the TaqMan Universal PCR Master Mix and an ABI Prism® 7900HT Fast Real-Time PCR System (Applied Biosystems). TaqMan Primers for GAPDH, ApoB, and 18S (Housekeeping gene) were purchased from Applied Biosystems. qPCR reactions were performed using manufacturer's protocol. Briefly, reaction volumes were incubated at 95° C. for 10 minutes to activate Taq polymerase followed by 40 cycles of 95° C. for 15 seconds for the denaturation of duplexes, and 60° C. for 1 minute to allow for elongation. The relative level of gene expression for each sample is calculated by the ΔΔC_{T} method as described for example in Livak and Schmittgen, 2001, “Analysis of relative gene expression data using real-time quantitative PCR and the 2(-Delta Delta C(T)) Method,” Methods 25, 402-8. Threshold values (C_{T}) for both the target gene (ApoB or GAPDH) and housekeeping gene (18S or GAPDH) were determined with the ABI PRISM® 7700 Sequence Detection System software. To determine gene silencing, C_{T} values for each sample were first normalized by subtracting the C_{T} values of the house keeping genes from each sample C_{T}. Gene expression levels were then determined by first determining the ΔΔC_{T} values obtained by subtracting the normalized C_{T} values for control groups (PBS) from the normalized C_{T} values of each test group sample. To ensure primer and total RNA quality, negative controls were analyzed by running samples without template. To ensure total RNA purity, samples were concurrently run during cDNA synthesis without the use of reverse transcriptase to ensure no genomic DNA contamination.

Statistical Analysis

To ensure statistical significance, all experiments were performed with at least 3 samples per group (n=3). Standard deviations were calculated for each group and analysis of statistical significance was determined using either a two-tailed Student's t-test or One-Way Analysis of Variance (ANOVA) with Tukey's test to compare between treatment groups. Significance was set for values where p<0.05. For in vivo studies, the numbers of mice in each group were determined by use of power analysis targeting a difference in means of 20% with a standard deviation of 10%, using an α=0.05 and a power of 0.80. This resulted in a sample size of 5 mice per group. All error bars represent standard deviation.

Silencing of GAPDH in Murine Lungs and Liver Following Intravenous Delivery of PEGylated Chitosan-IAA/siRNA Nanoparticles

In the past, intravenous delivery of siRNA molecules has been limited by serum instability, lack of intracellular and tissue penetration, and lack of targeting. The present in vivo experiments were conducted to determine the capability of PEGylated, non-targeted chitosan-IAA/siRNA nanoparticles to induce gene silencing in the lungs and liver following intravenous injection.

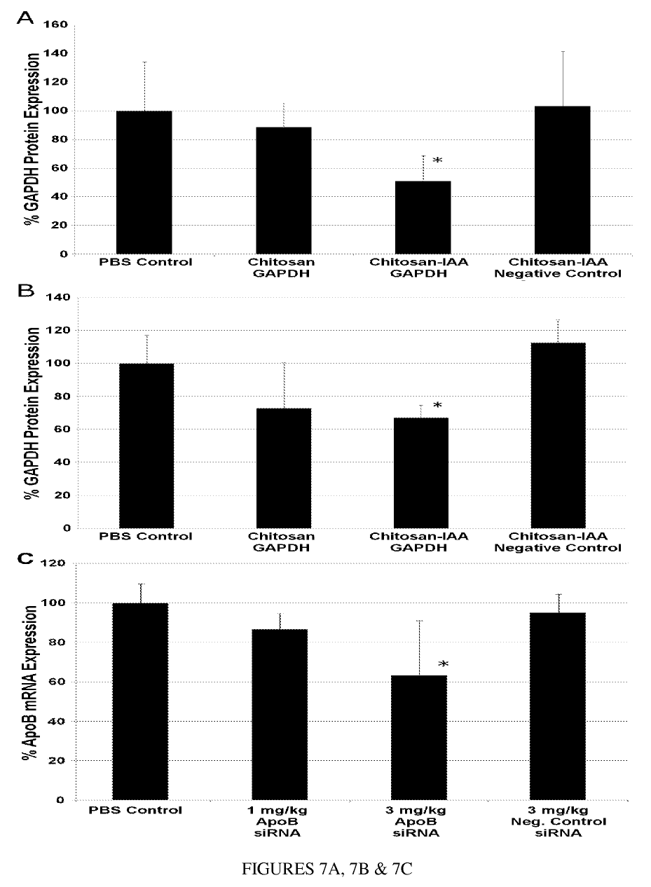

Female BALB/C mice (6-8 weeks of age) were injected intravenously via the tail vein with chitosan/siRNA and chitosan-IAA/siRNA (GAPDH) nanoparticles and the lungs and liver were analyzed for GAPDH silencing. The earlier characterized (Ambion positive control-target human, mouse and rat gene) hyper-potent GAPDH siRNA that was used in this study was been previously identified via extensive in vitro screening. To counteract potential serum-induced aggregation due to protein adsorption, PEG (mPEG-SVA) was conjugated to the nanoparticles at a ratio of 1:5 (PEG:NH₂). Initial studies were performed by injection of nanoparticles at a concentration of 1 mg/kg siRNA to each mouse and N/P ratio of 50. Three days following injection, mice were sacrificed and whole lungs and liver were harvested. Analysis of GAPDH protein expression in the lungs, demonstrated a significant gene silencing effect of up to ˜49% when siRNA was delivered using chitosan-IAA (FIG. 7A). Chitosan/siRNA nanoparticles mediated minimal gene silencing effect for GAPDH (˜11%) when administered via intravenous injection, producing an insignificant silencing effect as compared to control groups. Similar results were noted in C57BL/6J mice (6-9 weeks of age) following treatment with identical formulations.

In vivo gene silencing in the liver following intravenous administration of PEGylated nanoparticles was also evaluated. As shown in FIG. 7B, three days following injection, chitosan-IAA nanoparticles demonstrated significant gene silencing in the whole liver at ˜33% when compared to the PBS control group. While chitosan nanoparticles provided ˜27% average gene silencing effect following injection, high variability resulted in no statistically significant difference compared to the PBS control group. Similarly to previous studies, negative control siRNA delivered with chitosan-IAA nanoparticles demonstrated minimal gene silencing effect.

Dose-Dependent Gene Silencing of Apolipoprotein B (ApoB) in the Liver Following Treatment with Chitosan-IAA/siRNA Nanoparticles

Chitosan-IAA/siRNA nanoparticles were studied to further examine the effect of variable dose on gene silencing of apolipoprotein B, a carrier molecule for low-density cholesterol (LDL). Female BALB/C mice (n of at least 4 per group) were intravenously injected with nanoparticles prepared from chitosan-IAA and either ApoB-targeted Silencer® Select siRNA or a Negative Control siRNA (Ambion). Three ApoB siRNAs were evaluated in vitro, and the sequence that induced maximal knockdown of the mRNA target at lowest concentration was selected for in vivo studies (data not shown). Silencer® Select siRNAs are designed using a novel algorithm and incorporates limited number of LNA modifications that provide enhanced potency and superior specificity. Particles were prepared at an N/P ratio of 40 and surface modified with mPEG-SVA to improve serum stability. Mice were injected with final doses of 1 and 3 mg/kg siRNA via two injections over two consecutive days (200 μL/dose) through the tail vein. As shown in FIG. 7C, three days after final injection, gene expression levels determined by qPCR demonstrated gene silencing of ˜15% and ˜37% for 1 and 3 mg/kg siRNA doses, respectively. Mice that were administered a total 3 mg/kg siRNA dose demonstrated significant gene silencing of ApoB as compared to all control groups. Groups administered with 3 mg/kg doses of negative control siRNA nanoparticles demonstrated minimal to no silencing as expected.

Chitosan-IAA/siRNA-Mediated Silencing of GAPDH in Lungs Following Intranasal Administration

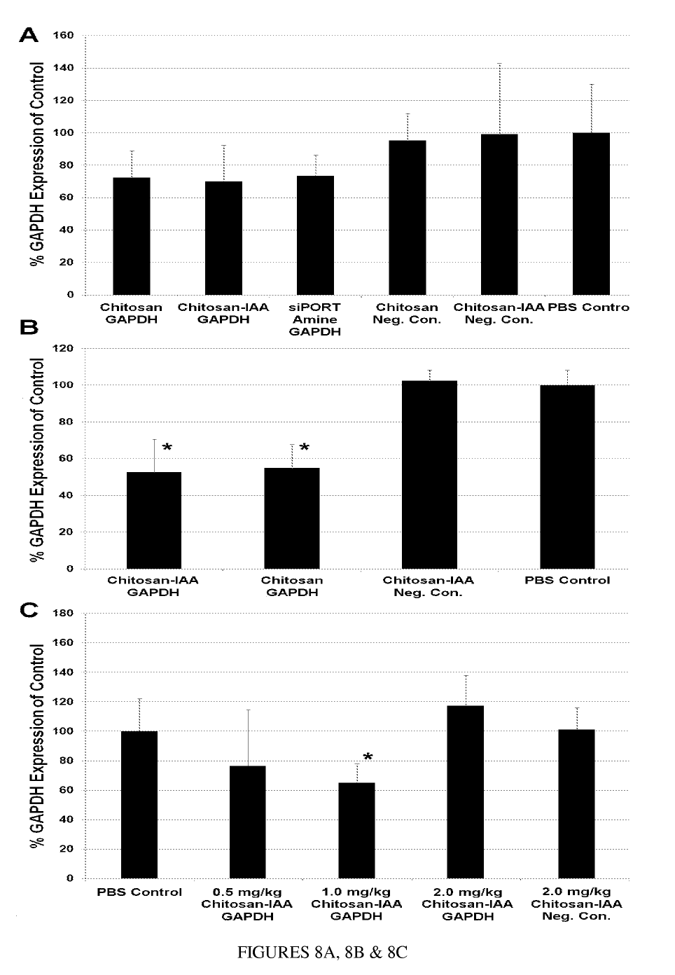

In vivo experiments were performed to compare the delivery efficiency of siRNA following intranasal administration with chitosan or chitosan-IAA/siRNA nanoparticles. Silencing of GAPDH expression in the lungs was evaluated at low siRNA doses (0.5 mg/kg single dose or three consecutive daily doses of 0.5 mg/kg). No observable adverse effects were noted in mice following administration of various nanoparticles formulations. 3 days post administration, treated mice were sacrificed and whole lungs were analyzed for GAPDH expression. Following a single low-dose injection in C57BL/6J mice, some GAPDH knockdown was observed (FIG. 8A) although the differences between the experimental and control groups were not statistically significant. Mice treated with chitosan-IAA/siRNA, chitosan/siRNA and siPORT Amine/siRNA all showed an average knockdown of 26-30% in the whole lungs.

Because single low-dose administrations did not show statistically significant knockdown, multiple low dose administrations were performed to determine enhancement in gene silencing. Mice (C57BL/6J) were treated with an identical dose (0.5 mg/kg siRNA) daily for three consecutive days. Treated animals were sacrificed and lungs were collected three days following the final administration and analyzed for GAPDH protein content. Use of multiple doses demonstrated significant gene silencing (47%) for chitosan-IAA/siRNA nanoparticles as is shown in FIG. 8B. Similarly, chitosan/siRNA nanoparticles elucidated 45% silencing of GAPDH. There were no significant differences between the modified and non-modified polysaccharide. The siPORT Amine nanoparticles demonstrated cytotoxicity in initial multiple dose studies, and therefore were not further used. In comparison, no silencing effect was noted for mice treated with negative control siRNA nanoparticles delivered with chitosan-IAA.

To study the effects of varying siRNA dosage following intranasal administration of chitosan-IAA/siRNA nanoparticles, siRNA doses of 0.5, 1.0, and 2.0 mg/kg of siRNA were evaluated in BALB/C mice. Higher doses of siRNA demonstrated no adverse affects on the mice following administration. As shown in FIG. 8C, GAPDH gene silencing (mRNA level) of up to 35% was demonstrated with a single dose when 1 mg/kg of Anti-GAPDH siRNA was delivered with chitosan-IAA. Low-dose samples (0.5 mg/kg) demonstrated similar silencing effects to earlier studies with gene silencing of ˜24%. The treatment of mice at the highest dose (2.0 mg/kg) for chitosan-IAA nanoparticles produced no silencing effect in mice when administered intranasally. As discussed below, this demonstrates a potential limitation, possibly due to the higher viscosity of the formulation, in the effective dosing range for chitosan-IAA mediated siRNA for intranasal delivery.

The present disclosure demonstrates in the above described example embodiments that chitosan-IAA/siRNA nanoparticles provide efficient gene silencing in lungs and liver following administration via intranasal and intravenous routes. Effective use of chitosan-IAA as an in vivo delivery vector for siRNA via both local (intranasal) and systemic (intravenous) delivery routes has been demonstrated and significant enhancement of siRNA-mediated gene silencing in both the liver and lungs.

While embodiments of this disclosure have been depicted, described, and are defined by reference to specific embodiments of the disclosure, such references do not imply a limitation of the disclosure, and no such limitation is to be inferred. The subject matter disclosed is capable of considerable modification, alteration, and equivalents in form and function, as will occur to those ordinarily skilled in the pertinent art and having the benefit of this disclosure. The depicted and described embodiments of this disclosure are examples only, and are not exhaustive of the scope of the disclosure. For example, one skilled in the art may readily imagine a variety of combinations of different types of nucleic acids or anionic agents and polysaccharides and reactive compounds and assembly of nanoparticles and perhaps also additional materials. Treatment of a variety of conditions may be also envisaged.

What is claimed is:

1. A drug delivery vehicle comprising:
a nanoparticle comprising:
a modified polysaccharide having a degree of substitution with at least one secondary amine or at least one tertiary amine; and
at least one therapeutic nucleic acid or at least one therapeutic anionic agent.

2. The drug delivery vehicle of claim 1, wherein the modified polysaccharide comprises at least one secondary amine and at least one tertiary amine.

3. The drug delivery vehicle of claim 1, wherein the modified polysaccharide comprises at least two secondary amines.

4. The drug delivery composition of claim 1, wherein the therapeutic nucleic acid is a polynucleotide, a DNA sequence, a DNA sequence encoding a therapeutic protein, an RNA sequence, a small interfering RNA (siRNA), a micro RNA (miRNA), an antisense oligonucleotide, a triplex DNA, a plasmid DNA (pDNA) or any combinations thereof.

5. The drug delivery composition of claim 1, wherein the therapeutic anionic agent is an anionic protein, an anionic glycosaminoglycan, an anionic peptide, an anionic hormone, an anionic biomolecule, an anionic small molecule agent or any combination thereof.

6. The drug deliver vehicle of claim 1, wherein the polysaccharide is selected from a group consisting of a chitosan, a dextran modified to comprise one or more primary amines, a glucosamine, hybrid polymers of any of the previous polymers, and any combinations thereof.

7. The drug deliver vehicle of claim 1, wherein the polysaccharide is a chitosan.

8. The drug delivery vehicle of claim 7, wherein the degree of substitution of the chitosan with at least one secondary amine or at least one tertiary amine is at least about 0.5% as determined by NMR analysis comprising evaluation of imidazole peaks.

9. The drug delivery vehicle of claim 7, wherein the degree of substitution of the chitosan with at least one secondary amine or at least one tertiary amine is from about 0.5% to about 3% as determined by NMR analysis comprising evaluation of imidazole peaks.

10. The drug delivery vehicle of claim 7, wherein the degree of substitution of the chitosan with at least one secondary amine or at least one tertiary amine is at least about 10% as determined by ninhydrin assay and NMR analysis.

11. The drug delivery vehicle of claim 7, wherein the degree of substitution of the chitosan with at least one secondary amine or at least one tertiary amine is from about 19.9% to about 30.2% as determined by ninhydrin assay and NMR analysis.

12. The drug delivery vehicle of claim 1, having an effective buffering capacity in aqueous solution from about pH 4.5 to about pH 8.5.

13. The drug delivery vehicle of claim 1, wherein the polysaccharide is at least 90% soluble in an aqueous solution at a pH greater than about 7.

14. The drug delivery vehicle of claim 1, further comprising polyethylene glycol (PEG).

15. A method for synthesizing a drug delivery vehicle comprising:
reacting at least one reactive compound and a polysaccharide to introduce at least one secondary amine or at least one tertiary amine onto the polysaccharide, thereby obtaining a modified polysaccharide having a degree of substitution with the secondary amine or the tertiary amine; and
complexing a therapeutic nucleic acid or a therapeutic anionic agent to the modified polysaccharide to form a drug delivery vehicle.

16. The method of claim 15, wherein the reacting comprises reacting the at least one reactive compound and the polysaccharide to introduce both at least one secondary amine and at least one tertiary amine onto the polysaccharide.

17. The method of claim 15, further comprising reacting the at least one reactive compound and the polysaccharide to introduce at least two secondary amines onto the polysaccharide.

18. The method of claim 15, wherein the therapeutic nucleic acid is a polynucleotide, a DNA sequence, a DNA sequence encoding a therapeutic protein, an RNA sequence, a small interfering RNA (siRNA), a micro RNA (miRNA), an antisense oligonucleotide, a triplex DNA, a plasmid DNA (pDNA) or any combinations thereof.

19. The method of claim 15, wherein the polysaccharide is selected from a group consisting of a chitosan, a dextran modified to comprise one or more primary amines, a glucosamine, hybrid polymers of any of the previous polymers or any combinations thereof.

20. The method of claim 15, wherein the polysaccharide is a chitosan.

21. The method of claim 15, wherein the reactive compound is selected from a group consisting of imidazole-4-acetic acid, arginine, histidine, polyarginine, polyhistidine or any combinations thereof.

22. The method of claim 15, wherein the reactive compound comprises an imidazole.

23. The method of claim 15, wherein reacting continues until the degree of substitution of the polysaccharide with the at least one secondary amine or the at least one tertiary amine is at least about 0.5% as determined by NMR analysis comprising evaluation of imidazole peaks.

24. The method of claim 15 further comprising formulating a nanoparticle.

25. The method of claim 24, further comprising adding polyethylene glycol groups on the nanoparticle.

26. A method for delivery of a therapeutic nucleic acid comprising:
administering to a patient in need thereof a pharmaceutical formulation comprising:
a nanoparticle comprising:
a modified polysaccharide having a degree of substitution with at least one secondary amine or at least one tertiary amine; and
at least one therapeutic nucleic acid.

27. The method of claim 26, wherein the nanoparticle further comprises polyethylene glycol (PEG).

28. The method of claim 26, wherein the administering is by oral ingestion, sublingual administration, intranasal administration, intramuscular injection, subcutaneous injection, parenteral administration, intrabiliary administration, topical application, intravenous administration, intraperitoneal administration, subcutaneous administration, intrathecal administration, injection to spinal cord, intramuscular administration, intraarticular administration, portal vein injection or intratumoral administration.

29. The method of claim 26, wherein the administering comprises intranasal or intravenous administration.

30. The method of claim 26, wherein the therapeutic nucleic acid is a polynucleotide, a DNA sequence, a DNA sequence encoding a therapeutic protein, an RNA sequence, a small interfering RNA (siRNA), a micro RNA (miRNA), an antisense oligonucleotide, a triplex DNA, a plasmid DNA (pDNA) or any combinations thereof.

31. A method of reducing or ameliorating development of heart disease in a subject by reducing cholesterol in the subject comprising:
administering to the subject a pharmaceutical formulation comprising:
a) a chitosan-IAA having a degree of substitution of at least one secondary amine or at least one tertiary amine; and
b) an anti-ApoB siRNA.
